(19) United States
(12) Patent Application Publication
(10) Pub. No.: US 2021/0189490 A1
(43) Pub. Date: Jun. 24, 2021

(54) MicroRNA Transcriptional Signature (miR-TS) Evaluation of Stem Cells

(71) Applicant: Lundquist Institute for Biomedical Innovation at Harbor-UCLA Medical Center, Torrance, CA (US)

(72) Inventor: Nestor F. Gonzalez-Cadavid, Pasadena, CA (US)

(21) Appl. No.: 17/193,637

(22) Filed: Mar. 5, 2021

Related U.S. Application Data
(63) Continuation of application No. PCT/US2019/054143, filed on Oct. 1, 2019.
(60) Provisional application No. 62/739,680, filed on Oct. 1, 2018.

(51) Int. Cl. C12Q 1/6881; C12Q 1/6883

(52) U.S. Cl. CPC C12Q 1/6881; C12Q 1/6883; C12Q 2600/106; C12Q 2600/178; C12Q 2600/158

(57) ABSTRACT

Aspects of the present disclosure include methods of evaluating stem cells. Aspects of the methods include obtaining a microRNA transcriptional signature (miR-TS), i.e., a microRNA transcriptional phenotype, for a stem cell and using the obtained miR-TS to evaluate the stem cell. Also provided are kits and compositions for practicing the subject methods. The methods and compositions find use in a variety of different applications, including diagnostic applications, therapeutic applications, research applications, and the like.

### CROSS REFERENCE TO RELATED APPLICATIONS

Pursuant to 35 U.S.C. § 119 (e), this application claims priority to the filing date of the U.S. Provisional Patent Application Ser. No. 62/739,680, filed Oct. 1, 2018; the disclosure of which application is herein incorporated by reference.

### INTRODUCTION

Stem cells are cells that have the ability both to self-renew, and to generate differentiated progeny (see Morrison et al. (1997) Cell 88:287-298). Generally, stem cells also have one or more of the following properties: an ability to undergo asynchronous, or asymmetric replication; i.e., where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages.

Stem cell implantation into various types of damaged tissues has emerged as a promising therapy for several human urological disorders based on solid experimental evidence in animal models, and specifically in sexual medicine for erectile dysfunction and Peyronie's disease. These studies have translated to therapy the extensive biological research showing the ability of stem cells to proliferate indefinitely and differentiate into the desired cell lines intended to replace the lost or damaged cells, or alternatively assuming that they may favorably change the damaged tissue composition through paracrine or juxtacrine effects. The main premise for their efficacy and safety is the belief, or rather the lack of contrary evidence, that the “immortal” stem cells are essentially resistant in their tissues of origin, and later in their sites of implantation, to the chronic noxious conditions and endogenous factors in many diseases, and even some physiological processes that damage the differentiated cells. This assumption extends to the tissue repair elicited by endogenous (rather than implanted) local or recruited stem cells, such as in the penile corpora cavernosa or tunica albuginea, where the potential damage to stem cells by the tissue milieu affected by the disease has not been studied.

### SUMMARY

Aspects of the present disclosure include methods of evaluating stem cells. Aspects of the methods include obtaining a microRNA transcriptional signature (miR-TS), i.e., a microRNA transcriptional phenotype, for a stem cell and using the obtained miR-TS to evaluate the stem cell. Also provided are kits and compositions for practicing the subject methods. The methods and compositions find use in a variety of different applications, including diagnostic applications, therapeutic applications, research applications, and the like.

Aspects of the invention solve a number of problems. In particular: stem cell therapy inefficacy, abnormal cell lineage commitment, and noxious side effects caused by stem cell damage, such as for stem cell autografts from and to long-term patients with type 2 diabetes, obesity, and dyslipidemia, to treat erectile dysfunction and other associated urological and vascular or neurological complications, such as limb ischemia or inadequate wound healing. More in general, for the stem cell treatment in these patients of other co-morbidities not necessarily resulting from diabetes/obesity, or even in patients with other chronic conditions, as aging, inflammation, cardiovascular disease, cancer, and excessive cytokine and (reactive oxygen species (ROS) release, likely to damage their own stem cells or the ones from other donors in their tissues of origin.

Embodiments of the invention include a diagnostic procedure to detect stem cell damage caused by prolonged exposure to chronic disease or aging milieu in the patient who is the donor and recipient of the autografts, or in other individual donors, or in stem cell banks. Such embodiments are based on the pioneering finding described herein that long term exposure of stem cells to a noxious milieu damages their tissue and functional repair capacity, imprinting them with an abnormal phenotype that is best defined by alterations of their microRNA transcriptional signatures (miR-TS), and can be reproduced in vitro with the serum of either recipient or donor subject.

The microRNA transcriptional phenotype biomarker assay described herein is sensitive, accurate, and can be used in a number of applications, such as: 1) to detect in vivo damage of stem cells, impairing their use as autografts or allografts, where use of damaged stem cells may be ineffective at best or very risky for the patient at worst; 2) to predict a defective self repair response of the injured tissues and related diseases or conditions by the patient own endogenous stem cells; 3) to test and evaluate in vitro methods and procedures to prevent or reverse the stem cell damage.

Except for autocrine stem cell senescence, little or no attention is given in stem cell therapy to the donor patient morbidities and co-morbidities that may affect the systemic milieu where the stem cells are immersed in their tissue of origin, because there is little or no consideration in clinical practice for the possibility that stem cells may be as sensitive to damage as the injured differentiated cells that they aim to replace. In the current invention we show that stem cell damage actually occurs in the context of dyslipidemia both in vivo and in vitro, and we developed an in vitro model and genomic tests to study its mechanism. Embodiments of the invention employ the miR-TS as a diagnostic and follow up tool applied to assess the damage and its possible spontaneous or induced reversal, since there is no prior literature in this respect.

Aspects of the invention include a novel diagnostic, preventive and/or follow up procedure for: 1) detecting damage to the tissue repair capacity of a subject's endogenous stem cells due to their long-term exposure to the respective systemic milieus in, or from: 1a) type-2 diabetic/obese/dyslipidemic subjects; or 1b) subjects with other chronic diseases or noxious conditions as aging; or 2) excluding the therapeutic implantation of damaged stem cells as: 2a) autografts in the same donor patients who are recipients of these stem cells; or as 2b) allografts in other compatible recipient patients; or 3) foreseeing the noxious effects on the implanted stem cells by the recipient systemic milieu represented by the serum of the recipient patients; or 4) studying and performing the potential in vivo and in vitro therapeutic reversal of the stem cell damage by biological and pharmacological agents; as well as combinations of any of the above.

The diagnostic, preventive and/or follow up procedure includes the determination, e.g., by next generation sequencing, RT/PCR, or other suitable protocol, e.g. sequencing protocol: 1) the microRNA (miR) global transcriptional signatures (miR-GTS) representing the levels of each of one or more miRs, or 2) miR individual transcriptional signatures (miR-ITS), representing the levels of selected miRs, that characterize the damaged stem cells in comparison to the normal stem cells. This is performed on any convenient sample, such as but not limited to: 1) the stem cells exposed in vivo to the subject's milieu, their exosomes, the unfractionated subjects blood sera, or the serum exosomes; or 2) the stem cells exposed in vitro to the subjects 2a) sera; or 2b) biological or pharmacological agents.

In addition the intact exosomes, or individual miRs, from normal stem cells, normal serum, or their respective total miRs in the normal miR-GTS may be 1) implanted in vivo in the recipient subject, to prevent endogenous or implanted stem cell damage, or 2) incubated in vitro with the in vivo damaged stem cells to reverse their damage for their subsequent use for implantation.

### BRIEF DESCRIPTION OF THE FIGURES

FIG. 1. A moderate hyperglycemia but intense dyslipidemia develops with age in male diabetic obese Zucker (OZ) rats in contrast to the related strain non-diabetic lean Zucker (LZ) rats. Animals were subjected to weekly evaluation of glucose, cholesterol, triglycerides, and insulin (not shown) in the serum from 12 to 28 weeks of age.

FIG. 2. Incubation of ED-MDSC with a higher level of aged OZ serum induced a considerable intracellular infiltration by fat globules. ED-MDSC were incubated with no addition (A), or with added 5% LZ serum (B) or 5% OZ serum (C) as in FIG. 2 Supplement, and stained with Oil Red O. Pictures were taken at 200×, but QIA was applied to multiple fields at 100× (D) Graph of red area (fat) per cell. ***p≤0.005 (CTR vs OZ); ^{∘∘∘}p≤0.005 (LZ vs OZ)

FIG. 3. Incubation of ED-MDSC with water-soluble preparations of the sodium salt of palmitic acid (PA) as representative of saturated free fatty acids, or cholesterol (CHOL), induced a milder infiltration of fat globules. ED-MDSC were incubated with no addition (A), or with added PA at 0.5 (B) or 1 mM (not shown), or added CHOL at 50 mg/dl (C), or 100 mg/dl (not shown) and stained with Oil Red O. Pictures were taken at 200×, but QIA was applied to multiple fields at 100× (D) Graph of red area (fat) per cell. ***p≤0.005 (CTR vs OZ)

FIG. 4. Incubation of ED-MDSC with 5% aged OZ serum induced moderate apoptosis. ED-MDSC were incubated with no addition (A), or with added 5% serum from aged LZ rats (B) or 5% serum from aged OZ rats (C) as in FIG. 2 and stained with the Tunel reaction. Pictures were taken at 200×, but QIA was applied to multiple fields at 100× (D). Graph of apoptotic index. ***p≤0.005 (CTR vs OZ); ^{∘∘}p≤0.01 (LZ vs OZ) The morphology of the control cells appears slightly different than in the previous figures stained for Oil Red O, but leaving aside the typical stem cell coexistence of morphological variants, is the different background color in both types of reactions what mostly creates this impression.

FIG. 5. Incubation of ED-MDSC with preparations of water-soluble palmitic acid (PA), or cholesterol (CHOL), induced considerable apoptosis. ED-MDSC were incubated with no addition (A), or with added PA at 0.5 (B), or added CHOL at 50 mg/dl (B) or 100 (C) and stained with the Tunel reaction. Pictures were taken at 200×, but QIA was applied to multiple fields at 100× (D) Graph of apoptotic index. ***p≤0.005 (CTR vs OZ); ^{∘∘∘}p≤0.005 (LZ vs OZ)

FIG. 6. Incubation of ED-MDSC with OZ serum caused a moderate decrease in cell replication and smooth muscle differentiation. ED-MDSC were incubated in duplicate with no addition, with added 0.5%, 2.5%, or 5% aged OZ or LZ serum, and subjected to western blot as indicated, with beta-actin as a housekeeping gene. See FIG. 7 for quantitative analysis.

FIG. 7. The quantitative image analysis (QIA) of the protein band densitometries in the incubations of ED-MDSC with OZ serum and palmitic acid sodium salt confirmed the visual inspection of the western blot images. The selected bands of the duplicate experiments seen in FIG. 6 and in FIG. 2 Supplement (single experiment with cholesterol excluded) were subjected to QIA and the means+/−SEM were statistically compared for variance in each type of incubations. *p≤0.05 compared to control

FIG. 8. Incubation of ED-MDSC with increasing aged OZ serum caused a concentration dependent expression of myostatin protein, a pro-lipofibrotic and muscle mass inhibitor, but the incubation with increasing glucose exerted an opposite effect. Incubations were performed on 6 well plates with no addition, or in duplicate with increasing concentrations of OZ or LZ serum (A), or of glucose either before or after 2 mM azacytidine for 2 days to stimulate stemness (C). Cell homogenates were subjected to western blot as indicated, with beta-actin as housekeeping gene, and densitometry was applied for the A samples (B). Other biomarkers for fibrosis, apoptosis, replication, and SMC differentiation were assayed in C, but no changes were observed (not shown). *p≤0.05 compared to control

FIG. 9. Incubation of muscle derived stem cells (MDSC) isolated from early diabetes OZ rats (ED-MDSC), with low levels of serum from aged OZ rats (OZ serum), induced a moderate infiltration by fat globules. ED-MDSC were incubated in DMEM-10% fetal calf serum receiving either 0.5% (A) or 2.5% (C) of serum from 24 weeks old aged LZ rats, or 0.5% (B) or 2.5% (D) of serum from age-matched OZ rats. At 4-5 days they were stained with Oil Red O. Pictures were taken at 200×

FIG. 10. Incubation of ED-MDSC with lipid factors caused a severe decrease in cell replication and smooth muscle differentiation. ED-MDSC were incubated in duplicate with no addition or with added 1 and 2 mM soluble sodium palmitate, and in a single experiment with 100, 200, and 400 mg/dl of cholesterol, and subjected to western blot as indicated, with beta-actin as a housekeeping gene. See FIG. 7 for quantitative analysis. *p≤0.05 compared to control

### DETAILED DESCRIPTION

Aspects of the present disclosure include methods of evaluating stem cells. Aspects of the methods include obtaining a microRNA transcriptional signature (miR-TS), i.e., a microRNA transcriptional phenotype, for a stem cell and using the obtained miR-TS to evaluate the stem cell. Also provided are kits and compositions for practicing the subject methods. The methods and compositions find use in a variety of different applications, including diagnostic applications, therapeutic applications, research applications, and the like.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As summarized above, methods of evaluating a stem cell are provided. As discussed above, stem cells are cells that have the ability both to self-renew, and to generate differentiated progeny (see Morrison et al. (1997) Cell 88:287-298). Generally, stem cells also have one or more of the following properties: an ability to undergo asynchronous, or asymmetric replication; i.e., where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages.

Stem cells include adult stem cells. Adult stem cells are also referred to as somatic stem cells. Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some embodiments, the stem cell is a hematopoietic stem cell (HSC). HSCs are mesoderm-derived cells that can be isolated from bone marrow, blood, cord blood, fetal liver and yolk sac. HSCs are characterized as CD34+ and CD3-. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other embodiments, the stem cell is a neural stem cell (NSC). Neural stem cells (NSCs) are capable of differentiating into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other embodiments, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

In other embodiments, the stem cell is muscle derived stem cell (MDSC). Muscle-derived stem cells (MDSC), also called muscle stem cells, include cells that are isolated as described in WO 99/56785 (University of Pittsburgh) and in U.S. Pat. Nos. 6,866,842 and 7,115,417 the contents of which are hereby incorporated by reference herein in their entireties. MDSCs are also described in Deasy et al., Muscle-derived stem cells: characterization and potential for cell-mediated therapy,” Blood Cells Mol Dis. 2001 September-October; 27(5):924-33.

Stem cells of interest include aged adult stem cells. For example, in some cases, an aged adult stem cell is an adult stem cell obtained from, or present in, a human individual greater than 50 years, greater than 55 years, greater than 60 years, greater than 65 years, greater than 70 years, greater than 75 years, greater than 80 years, greater than 85 years, or greater than 90 years of age. In some cases, an aged adult stem cell is an adult stem cell obtained from, or present in, a human individual who is from 50 years to 55 years, from 55 years to 60 years, from 60 years to 65 years, from 65 years to 70 years, from 70 years to 75 years, from 75 years to 80 years, from 80 years to 85 years, or from 85 years to 90 years of age.

The stem cells that are subject of the disclosed methods may be obtained from any suitable host. As used herein, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal.

In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

Aspects of the methods include obtaining a microRNA transcriptional phenotype (also referred to herein as a microRNA Transcriptional signature (miR-TS) for a sample comprising stem cell, or a component thereof, e.g., a vesicular component, such as an exosome that is to be evaluated. Depending on the particular method being practiced, the sample may vary. In some instances, the sample includes a stem cell, e.g., where the sample is a potentially therapeutic stem cell composition, e.g., obtained from a donor or other source, e.g., stem cell bank. Alternatively, the sample may be a sample from a participant in a stem cell procedure, e.g., a donor or recipient, where the sample may be a stem cell composition obtained from the participant, a blood sample, e.g., whole blood or a fraction thereof, e.g., serum, a vesicular, e.g., exosome, sample, etc.

The microRNA transcriptional phenotype may that is obtained for the sample may vary. In some embodiments, the microRNA transcriptional phenotype may include expression data for a single microRNA of interest. In yet other embodiments, the microRNA transcriptional phenotype may include expression data for 2 or more microRNAs of interest, such as 3 or more, 4 or more, 5 or more, 10 or more, 15 or more, 20 or more, 25 or more, 50 or more, 75 or more or 100 or more microRNAs of interest. In some instances, the microRNA transcriptional phenotype is a global transcriptional phenotype, by which is meant that the expression level of 20% or more, such as 30% or more, 40% or more, 50% or more, 60% or more, 70% or more, 80% or more, 90% or more, 95% or more, 99% or more, including all of the distinct microRNA species in the sample.

microRNAs that may be assayed in a given method may vary. In some instances, the microRNAs of interest include, but are not limited to: myostatin related microRNAs, such as, but not limited to, those myostatin related micro-RNAs listed in Table 3; myostatin un-related microRNAs, such as but not limited to, those myostatin un-related micro-RNAs listed in Table 4; the microRNAs listed in Table 5; the microRNAs listed in Table 6; the microRNAs listed in Table 7; and the microRNAs listed in Table 8. In a given method, the expression level of 1 or more, including 2 or more, such as 3 or more, 4 or more, 5 or more, 10 or more, 15 or more, 20 or more, 25 or more, including all of the microRNAs from any Tables 3 to 10 may be evaluated.

The level of a miRNA gene product in a sample can be measured using any technique that is suitable for detecting RNA expression levels in a biological sample. Suitable techniques for determining RNA expression levels in a biological sample are well known to those of skill in the art. These include, for example, Northern blot analysis, RT-PCR, in situ hybridization, next generation sequencing, etc.

The nucleic acid to be detected may be from a biological sample such as a tissue sample and the like. Various methods of extraction are suitable for isolating the DNA or RNA. Suitable methods include phenol and chloroform extraction, TCA/acetone precipitation, ethanol precipitation, Column purification, Silica gel membrane purification, etc. See Maniatis et al., Molecular Cloning, A Laboratory Manual, 2d, Cold Spring Harbor Laboratory Press, pp. 16-54 (1989). Numerous commercial kits also yield suitable DNA and RNA including, but not limited to, QIAamp™ mini blood kit, Agencourt Genfind™ Roche Cobas™, Roche MagNA Pure™ or phenol:chloroform extraction using Eppendorf Phase Lock Gels™ and the NucliSens extraction kit (Biomerieux, Marcy l'Etoile, France). TCA/acetone precipitation, ethanol precipitation, Column purification, Silica gel membrane purification, PureYield RNA Midiprep (Promega), PolyATtract System 1000 (Promega), Maxwell 16 System (Promega), SV Total RNA Isolation (Promega), geneMAG-RNA/DNA kit (Chemicell), TRI Reagent (Ambion), RNAqueous Kit (Ambion), ToTALLY RNA Kit (Ambion), Poly(A)Purist Kit (Ambion) and any other methods, commercially available or not, known to the skilled person. The RNA may be further amplified, cleaned-up, concentrated, DNase treated, quantified or otherwise analyzed or examined such as by agarose gel electrophoresis, absorbance spectrometry or Bioanalyzer analysis (Agilent) or subjected to any other post-extraction method known to the skilled person. Methods for extracting and analyzing an RNA sample are disclosed in Molecular Cloning, A Laboratory Manual (Sambrook and Russell (ed.), 3^{rd} edition (2001), Cold Spring Harbor Laboratory Press, Cold Spring Harbor, N.Y., USA.

In one embodiment, the level of at least one miRNA gene product is detected using Northern blot analysis. For example, total RNA can be purified from a sample in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7.

Suitable probes (e.g., DNA probes or RNA probes) for Northern blot hybridization of a given miRNA gene product can be produced from the known nucleic acid sequences and include, but are not limited to, probes having at least about 70%, 75%, 80%, 85%, 90%, 95%, 98% or 99% complementarity to a miRNA gene product of interest, as well as probes that have complete complementarity to a miRNA gene product of interest. Methods for preparation of labeled DNA and RNA probes, and the conditions for hybridization thereof to target nucleotide sequences, are described in Molecular Cloning: A Laboratory Manual, J. Sambrook et al., eds., 2nd edition. Cold Spring Harbor Laboratory Press, 1989, Chapters 10 and 11.

For example, the nucleic acid probe can be labeled with, e.g., a radionuclide, such as ³H, ³²P, ³³P, ¹⁴C, or ³⁵S; a heavy metal; a ligand capable of functioning as a specific binding pair member for a labeled ligand (e.g., biotin, avidin or an antibody); a fluorescent molecule; a chemiluminescent molecule; an enzyme or the like. Probes can be labeled to high specific activity by either the nick translation method or by the random priming method. Autoradiographic detection of hybridization can then be performed by exposing hybridized filters to photographic film. Densitometric scanning of the photographic films exposed by the hybridized filters provides an accurate measurement of miRNA levels. Using another approach, miRNA gene transcript levels can be quantified by computerized imaging systems.

In one embodiment, the miRNA is detected using a nucleic acid amplification process. Nucleic acid extracted from a sample can be amplified using nucleic acid amplification techniques well known in the art. Byway of example, but not byway of limitation, these techniques can include the polymerase chain reaction (PCR), reverse transcriptase polymerase chain reaction (RT-PCR), nested PCR, ligase chain reaction. See Abravaya, K., et al., Nucleic Acids Research, 23:675-682, (1995), branched DNA signal amplification, Urdea, M. S., et al., AIDS, 7 (suppl 2):S11-S 14, (1993), amplifiable RNA reporters, Q-beta replication, transcription-based amplification, boomerang DNA amplification, strand displacement activation, cycling probe technology, isothermal nucleic acid sequence based amplification (NASBA). See Kievits, T. et al., J Virological Methods, 35:273-286, (1991), Invader Technology, or other sequence replication assays or signal amplification assays may also be used.

Some methods employ reverse transcription of RNA to cDNA. The method of reverse transcription and amplification may be performed by previously published or recommended procedures. Various reverse transcriptases may be used, including, but not limited to, MMLV RT, RNase H mutants of MMLV RT such as Superscript and Superscript II (Life Technologies, GIBCO BRL, Gaithersburg, Md.), AMV RT, and thermostable reverse transcriptase from Thermus thermophiles. For example, one method which may be used to convert RNA to cDNA is the protocol adapted from the Superscript 11 Preamplification system (Life Technologies, GIBCO BRL, Gaithersburg, Md.; catalog no. 18089-011), as described by Rashtchian, A., PCR Methods Applic., 4:S83-S91, (1994).

In a suitable embodiment. PCR is used to amplify a target sequence of interest-PCR is a technique for making many copies of a specific template DNA sequence. The reaction consists of multiple amplification cycles and is initiated using a pair of primer sequences that hybridize to the 5′ and 3′ ends of the sequence to be copied. The amplification cycle includes an initial denaturation, and typically up to 50 cycles of annealing, strand elongation and strand separation (denaturation). In each cycle of the reaction, the DNA sequence between the primers is copied. Printers can bind to the copied DNA as well as the original template sequence, so the total number of copies increases exponentially with time. PCR can be performed as according to Whelan et al., J of Clin Micro, 33(3):556-561 (1995). Briefly, a PCR reaction mixture includes two specific primers, dNTPs, approximately 0.25 U of Taq polymerase, and 1×PCR Buffer.

The skilled artisan is capable of designing and preparing primers that are appropriate for amplifying a target or marker sequence. The length of the amplification primers depends on several factors including the nucleotide sequence identity and the temperature at which these nucleic acids arc hybridized or used during in vitro nucleic acid amplification. The considerations necessary to determine a preferred length for an amplification primer of a particular sequence identity are well-known to a person of ordinary skill. For example, the length of a short nucleic acid or oligonucleotide can relate to its hybridization specificity or selectivity.

In some embodiments, the amplification may include a labeled primer or probe, thereby allowing detection of the amplification products corresponding to that primer or probe. In particular embodiments, the amplification may include a multiplicity of labeled primers or probes; such primers may be distinguishably labeled, allowing the simultaneous detection of multiple amplification products. Oligonucleotide probes can be designed which are between about 10 and about 100 nucleotides in length and hybridize to the amplified region. Oligonucleotides probes are preferably 12 to 70 nucleotides; more preferably 15-60 nucleotides in length; and most preferably 15-25 nucleotides in length. The probe may be labeled.

In one embodiment, a primer or probe is labeled with a fluorogenic reporter dye that emits a detectable signal. While a suitable reporter dye is a fluorescent dye, any reporter dye that can be attached to a detection reagent such as an oligonucleotide probe or primer is suitable for use in the invention. Such dyes include, but are not limited to, Acridine. AMCA, BODIPY, Cascade Blue, Cy2, Cy3, Cy5, Cy7, Edans, Eosin, Erythrosin, Fluorescein, 6-Fam, Tet, Joe, Hex, Oregon Green, Rhodamine, Rhodol Green, Tamra, Roz, and Texas Red.

In yet another embodiment, the detection reagent may be further labeled with a quencher dye such as Tamra, Dabcyl, or Black hole Quencher™ (BHQ), especially when the reagent is used as a self-quenching probe such as a TaqMan™ (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

Nucleic acids may be amplified prior to detection or may be detected directly during an amplification step (i.e., “real-time” methods). For example, amplified fragments may be detected using standard gel electrophoresis methods. In some embodiments, amplified fractions are separated on an agarose gel and stained with ethidium bromide by methods known in the art to detect amplified fragments. In some embodiments, the target sequence is amplified using a labeled primer such that the resulting amplicon is detectably labeled. In some embodiments, the primer is fluorescently labeled.

In one embodiment, detection of a miRNA, such as a nucleic acid from an a miR-16 or miR-199a, is performed using the TaqMan™ assay, which is also known as the 5′ nuclease assay (U.S. Pat. Nos. 5,210,015 and 5,538,848). The TaqMan™ assay detects the accumulation of a specific amplified product during PCR. The TaqMan™ assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target-containing template which is amplified during PCR.

TaqMan™ primer and probe sequences can readily be determined using the nucleic acid sequence information of the miRNA of interest. A number of computer programs, such as Primer Express (Applied Biosystems, Foster City, Calif.), can be used to rapidly obtain optimal primer/probe sets. It will be apparent to one of skill in the art that such primers and probes for detecting the target nucleic acids are useful in diagnostic assays for neoplastic disorders, such as HCC, and can be readily incorporated into a kit format. The present invention also includes modifications of the TaqMan™ assay well known in the art such as the use of Molecular Beacon probes (U.S. Pat. Nos. 5,118,801 and 5,312,728) and other variant formats (U.S. Pat. Nos. 5,866,336 and 6,117,635).

In an illustrative embodiment, real time PCR is performed using TaqMan™ Assays in combination with a suitable amplification/analyzer such as the ABI Prism™ 7900HT Sequence Detection System. The ABI PRISM™ 7900HT Sequence Detection System is a high-throughput real-time PCR system that detects and quantitates nucleic acid sequences. Real-time detection on the ABI Prism 7900HT or 7900HT Sequence Detector monitors fluorescence and calculates Rn during each PCR cycle. The threshold cycle, or Ct value, is the cycle at which fluorescence intersects the threshold value. The threshold value is determined by the sequence detection system software or manually. The Ct can be correlated to the initial amount of nucleic acids or number of starting cells using a standard curve.

In one embodiment, TaqMan™ MicroRNA Assays are used to detect the miRNA. TaqMan™ MicroRNA Assays arc predesigned assays that are available for the majority of content found on the miRBase miRNA sequence repository. In another embodiment, the mirVana™ qRT-PCR miRNA Detection Kit (Ambion) is a used to detect and quantify the miRNA. This is a quantitative reverse transcription-PCR (qRT-PCR) kit enabling sensitive, rapid quantification of miRNA (miRNA) expression from total RNA samples.

As a quality control measure, an internal amplification control may be included in one or more samples to be extracted and amplified. The skilled artisan will understand that any detectable sequence that is not typically present in the sample can be used as the control sequence. A control sequence can be produced synthetically. If PCR amplification is successful, the internal amplification control amplicons can then be detected. Additionally, if included in the sample prior to purification of nucleic acids, the control sequences can also act as a positive purification control.

A Microfluidic card allows high throughput, parallel analysis of mRNA or miRNA expression patterns, and allows for a quick and cost-effective investigation of biological pathways. The microfluidic card may be a piece of plastic that is riddled with micro channels and chambers filled with the appropriate probes. A sample in fluid form is injected into one end of the card, and capillary action causes the fluid sample to be distributed into the microchannels. The microfluidic card is then placed in an appropriate device for processing the card and reading the signal. Any commercially available (predesigned or custom-made) microfluidic card may be used. Said microfluidic card may comprise a number of probes and/or primers for analysing the expression of a number of miRNAs, such as between 1-10 miRNAs, for example 10-20 miRNA, such as between 20-30 miRNAs, for example 30-40 miRNA, such as between 40-50 miRNAs, for example 50-100 miRNA, such as between 100-200 miRNAs, for example 200-300 miRNA, such as between 300-400 miRNAs, for example 400-500 miRNA, such as between 500-1000 miRNAs. In one embodiment, the microfluidic card is TaqMan Array Human MicroRNA A+B Cards V2.0 (Applied Biosystems).

In yet other embodiments, the isolated RNA may be analyzed by microarray analysis. In one embodiment, the expression level of one or more miRNAs is determined by the microarray technique. A microarray is a multiplex technology that consists of an arrayed series of thousands of microscopic spots of DNA oligonucleotides or antisense miRNA probes, called features, each containing picomoles of a specific oligonucleotide sequence. This can be a short section of a gene or other DNA or RNA element that are used as probes to hybridize a DNA or RNA sample (called target) under high-stringency conditions. Probe-target hybridization is usually detected and quantified by fluorescence-based detection of fluorophore-labeled targets to determine relative abundance of nucleic acid sequences in the target. In standard microarrays, the probes are attached to a solid surface by a covalent bond to a chemical matrix (via epoxy-silane, amino-silane, lysine, polyacrylamide or others). The solid surface can be glass or a silicon chip, in which case they are commonly known as gene chip. DNA arrays are so named because they either measure DNA or use DNA as part of its detection system. The DNA probe may however be a modified DNA structure such as LNA (locked nucleic acid). In one embodiment, the microarray analysis is used to detect microRNA, known as microRNA or miRNA expression profiling. The microarray for detection of microRNA may be a microarray platform, wherein the probes of the microarray may be comprised of antisense miRNAs or DNA oligonucleotides. In the first case, the target is a labelled sense miRNA sequence, and in the latter case the miRNA has been reverse transcribed into cDNA and labelled. The microarray for detection of microRNA may be a commercially available array platform, such as NCode miRNA Microarray Expression Profiling (Invitrogen), miRCURY LNA microRNA Arrays (Exiqon), microRNA Array (Agilent), micro Paraflo Microfluidic Biochip Technology (LC Sciences), MicroRNA Profiling Panels (Illumina), Geniom Biochips (Febit Inc.), microRNA Array (Oxford Gene Technology), Custom AdmiRNA profiling service (Applied Biological Materials Inc.), microRNA Array (Dharmacon—Thermo Scientific), LDA TaqMan analyses (Applied Biosystems), Taqman microRNA Array (Applied Biosystems) or any other commercially available array. Microarray analysis may comprise all or a subset of the steps of RNA isolation, RNA amplification, reverse transcription, target labelling, hybridisation onto a microarray chip, image analysis and normalisation, and subsequent data analysis; each of these steps may be performed according to a manufacturers protocol. It follows, that any of the methods as disclosed herein above may further comprise one or more of the steps of: i) isolating miRNA from a sample, ii) labelling of said miRNA, iii) hybridizing said labelled miRNA to a microarray comprising miRNA-specific probes to provide a hybridization profile for the sample, iv) performing data analysis to obtain a measure of the miRNA expression profile of said sample. In another embodiment, the microarray for detection of microRNA is custom made. A probe or hybridization probe is a fragment of DNA or RNA of variable length, which is used to detect in DNA or RNA samples the presence of nucleotide sequences (the target) that are complementary to the sequence in the probe. One example is a sense miRNA sequence in a sample (target) and an antisense miRNA probe. The probe thereby hybridizes to single-stranded nucleic acid (DNA or RNA) whose base sequence allows probe-target base pairing due to complementarity between the probe and target. To detect hybridization of the probe to its target sequence, the probe or the sample is tagged (or labelled) with a molecular marker. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in microarrays refer to nucleotide sequences covalently attached to an inert surface, such as coated glass slides, and to which a mobile target is hybridized. Depending on the method the probe may be synthesized via phosphoramidite technology or generated by PCR amplification or cloning (older methods). To design probe sequences, a probe design algorithm may be used to ensure maximum specificity (discerning closely related targets), sensitivity (maximum hybridization intensities) and normalized melting temperatures for uniform hybridization.

In yet another embodiment, the isolated RNA is analyzed by nuclease protection assay. Nuclease protection assay is a technique used to identify individual RNA molecules in a heterogeneous RNA sample extracted from cells. The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

In yet other embodiments, the isolated RNA is analyzed using a next generation sequencing (NGS) protocol. As such, the methods for detecting miRNAs can also include hybridization-based technology platforms and massively parallel next generation small RNA sequencing that allow for detection of multiple microRNAs simultaneously. High-throughput NGS technologies, for instance, can be used to assay entire sets of RNA transcripts within biological samples, and can be used to compare RNA transcription profiles between biological samples. Microarrays and other screening technologies such as NGS may measure the presence/absence of a miRNA in a sample; sequence changes in a particular miRNA; the number of miRNA expressed below and/or above a certain concentration threshold in a sample; or an assessment of the relative or absolute amount of a particular miRNA in a sample. One commercially-available hybridization-based technology utilizes a sandwich hybridization assay with signal amplification provided by a labeled branched DNA (Panornics). Another hybridization-based technology is available from Nanostring Technology (nCounter miRNA Expression Assay), where multiple miRNA sequences are detected and distinguished with fluorescently-labeled sequence tags. Examples of next-generation sequencing are available from Life Technologies (SOLiD platform) and Illumina, Inc. (e.g., Illumina HumanHT-12 bead arrays).

Statistical methods can be used to set thresholds for determining when the level in a subject can be considered to be different than or similar to a reference level. In addition, statistics can be used to determine the validity of the difference or similarity observed between a sample miRNA level and the reference level. Useful statistical analysis methods are described in L. D. Fisher & G. vanBelle, Biostatistics: A Methodology for the Health Sciences (Wiley-Interscience, N Y, 1993). For instance, confidence (“p”) values can be calculated using an unpaired 2-tailed t test, with a difference between groups deemed significant if the p value is less than or equal to 0.05. As used herein a “confidence interval” or “Cl” refers to a measure of the precision of an estimated or calculated value. The interval represents the range of values, consistent with the data that is believed to encompass the “true” value with high probability (usually 95%). The confidence interval is expressed in the same units as the estimate or calculated value. Wider intervals indicate lower precision; narrow intervals indicate greater precision. Preferred confidence intervals of the invention are 90%, 95%, 97.5%, 98%, 99%, 99.5%, 99.9% and 99.99%. A “p-value” as used herein refers to a measure of probability that a difference between groups happened by chance. For example, a difference between two groups having a p-value of 0.01 (or p=0.01) means that there is a 1 in 100 chance the result occurred by chance. Preferred p values are 0.1, 0.05, 0.025, 0.02, 0.01, 0.005, 0.001, and 0.0001. Confidence intervals and p-values can be determined by methods well-known in the art. See, e.g., Dowdy and Wearden, Statistic for Research, John Wiley & Sons, New York, 1983.

On linear model for assessing differential expression in microarray experiments: Smith G K (2004) “Linear models and empirical bayes method for assessing differential expression in microarray experiments” Statistical Applications in Genetics and Molecular Biology. For AUC calculation: Mason S J and Graham N E (1982) “Areas beneath the relative operating characteristics (ROC) and relative operating levels (ROL) curves: Statistical significance and interpretation,” Q. J. R. Meteorol. Soc. textbf30 291-303. Multiple algorithms program for marker combination selection: An R based program with nine algorithms including random forest, ada boosting, svm, bagging, logistic regression, lasso, matt, cart, ctree is available, for example, as open-source software from the R Foundation. Random forests were also conducted according to Breiman, L. (2001), Random Forests, Machine Learning 45(1), 5-32. Sec also Breiman, L (2002), “Manual On Setting Up, Using, And Understanding Random Forests V3.1.

One may seek levels that are lower or higher than a control. The term “elevated levels” or “higher levels” as used herein refers to levels of a miRNA that are higher than what would normally be observed in a comparable sample from control or normal subjects or normal tissue from the patient (i.e., a reference value). Similarly, “reduced levels” or “lower levels” as used herein refer to levels of that are lower than what would normally be observed in a comparable sample from control or normal subjects, or normal tissue from the patient (i.e., a reference value). In some embodiments, “control levels” (i.e., normal levels) refer to a range of miRNA levels that would be normally be expected to be observed in undamaged stem cells. A control level may be used as a reference level for comparative purposes. The ranges accepted as outside “control levels” are dependent on a number of factors. For example, one laboratory may routinely determine the level of circulating miRNA in a sample that is different than the miRNA obtained for the same sample by another laboratory. Also, different assay methods may achieve different value ranges. Value ranges may also differ in various sample types, for example, different body fluids or by different treatments of the sample. One of ordinary skill in the art is capable of considering the relevant factors and establishing appropriate reference ranges for “control values” and “elevated/reduced values” of the present invention. For example, a series of samples from control subjects and subjects diagnosed with melanoma can be used to establish ranges that are “normal” or “control” levels and ranges that are “elevated” or “reduced” than the control range.

The level of one or more miRNAs measured in the test sample is normalized, such as by comparison to an internal reference nucleic acid, e.g., U44 or small RNA U6. The levels of the one or more miRNAs may then be compared to a reference value to determine if the levels of the one or more miRNAs are elevated or reduced relative to the reference value. Typically, the reference value is the level measured in a comparable sample of healthy stem cells.

As summarized above, the obtained microRNA transcriptional phenotype is employed to evaluate a stem cell and/or source environment thereof. The evaluation of the stem cell that may be made may be an assessment, determination or inference about the stem cell, or a stem cell sample from which the assays stem cell or derivate thereof (e.g., exosome) has been obtained. The evaluation may be an may be an assessment, determination or inference regarding where the stem cell is damaged or undamaged. A stem cell may be considered damaged if one or more of its normal functions is impaired, such as its restorative capacity, and the like. In some instances, the evaluation is an assessment, determination or inference regarding the type of damage that the stem cell has undergone. In some instances, the type of damage that is detected is damage resulting from environmental exposure. An example of environmental exposure damage that may be detected is damage resulting from the source milieu of the stem cell, such as the physiological state of the donor of the stem cell, e.g., whether the donor of the stem cell is aged, diseased, or otherwise provides a noxious milieu, etc. In some instances, the damage that is detected is damage resulting from a diseased state, where diseased states include, but are not limited to: diabetes, .e.g., Type 2 diabetes, obesity, and the like. In some instances, the dame that is detected is damage resulting from a phenotypic characteristic of the source organisms, where examples of phenotypic characteristics include, but are not limited to: dyslipidemia, _ and the like.

Another example of environmental exposure damage is damage resulting from storage conditions of a sample that includes a stem cell of interest. For example, damaged stem cells from a stem cell bank may be detected using methods of invention. The damage to such cells may be damage resulting from storage conditions of the stem cell bank, including improper storage conditions, such as storage outside of a desired temperature range, storage in improper containers, storage that exposed the stem cells to contamination, etc. Stem cells from any stem cell bank may be evaluated using methods of the invention, where stem cell banks include, but are not limited, those described in published United States Patent Application Nos. 20030215942, 20040091936, 20050276792, 20080227197, 20120046968, 20130052169, 20130276154,

20150191694 and 20190225937, the disclosures regarding stem cell banks and preparation/use thereof in these published applications are herein incorporated by reference.

In some instances, the evaluating includes making an assessment, determination or inference about a stem cell's therapeutic capacity. As such, the evaluation may include making an assessment, determination or inference that the stem cell has or not have therapeutic utility in a given therapeutic application. In other words, the evaluation may include making an assessment, determination or inference that a stem cell will or will not be use in a given therapeutic application. The particular therapeutic activity that a stem may be determined to possess or lack in a given embodiment may vary, where examples of therapeutic activity include, but are not limited to, tissue regenerative activity, and the like.

Where the sample from which the assayed stem cell is a sample that includes a plurality of stem cells, such as a stem cell composition obtained from a donor, stem cell bank, patient to be treated, etc., embodiments of the methods may include making a determination that the stem cell composition is suitable for further use, e.g., in a therapeutic application, such as an application where the composition is administered to a subject for therapeutic purposes. For example, in some instances one or more stem cells may be obtained from a potentially therapeutic stem cell composition and the microRNA transcriptional phenotype obtained therefore. Where the obtained microRNA transcriptional phenotype is indicative of cell stem cell damage, e.g., because it matches the transcriptional phenotype of a damaged reference stem cell, the stem cell composition may be determined to be unsuitable for therapeutic use, and therefore not employed in a therapeutic application, such as administration to a subject for therapeutic purposes. In such instances, the methods may include discarding the stem cell composition that has been determined to be unsuitable for therapeutic purposes. Alternatively, where the obtained microRNA transcriptional phenotype is indicative of an undamaged cell stem cell, e.g., because it matches the transcriptional phenotype of an undamaged reference stem cell, the stem cell composition may be determined to be suitable for therapeutic use, and therefore employed in a therapeutic application, such as administration to a subject for therapeutic purposes. In such instances, the methods may include employing the stem cell composition in a therapeutic application, e.g., by administering the composition to a subject in need thereof.

Instead of discarding a stem cell composition that has been determined to be damaged, the stem cell composition may be contacted with a restorative agent to restore that desired activity, e.g., therapeutic capacity of the stem cell composition. The restorative agent may vary as desired. Restorative agents that may be employed include biological restorative agents, such as but not limited to: nucleic acids, proteins, undamaged stem cells or derivatives thereof, e.g., vesicles, such as exosomes, etc. In those instances where the restorative agent comprises a stem cell or derivative thereof, e.g., exosome, the methods may include obtaining the restorative agent from a suitable source, such as a healthy donor (including a donor having stem cells that have been determined by the methods of the invention to be undamaged), a stem cell bank, etc. Restorative agents that may be employed also include small molecule restorage agents. Naturally occurring or synthetic small molecule compounds of interest include numerous chemical classes, such as organic molecules, e.g., small organic compounds having a molecular weight of more than 50 and less than about 2,500 daltons. Candidate agents comprise functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols described below. Contact of the stem cell composition with the restorative agent may occur in vitro, e.g., prior to administration of the composition to a subject in need thereof, or in vivo, e.g., prior to obtaining the composition from the source, e.g., where the source is the subject in need of the composition (such as in autograph application) or the source is a donor distinct from the subject in need of the composition (such as in an allograph application).

As indicated above, methods of the invention, such as therapeutic methods, may include administering a stem cell composition that has been determined to be undamaged to a subject in need thereof. Cell populations and related compositions may be provided to a patient by a variety of different means. In certain embodiments, they are provided locally, e.g., to a site of actual or potential injury. In one embodiment, they are provided using a syringe to inject the compositions at a site of possible or actual injury or disease. In other embodiments, they are provided systemically. In one embodiment, they are administered to the bloodstream intravenously or intra-arterially. The particular route of administration will depend, in large part, upon the location and nature of the disease or injury being treated or prevented. Accordingly, the invention includes providing a cell population or composition of the invention via any known and available method or route, including but not limited to oral, parenteral, intravenous, intra-arterial, intranasal, and intramuscular administration.

The development of suitable dosing and treatment regimens for using the cell populations and compositions described herein in a variety of treatment regimens, including e.g., oral, parenteral, intravenous, intranasal, and intramuscular administration and formulation, will again be driven in large part by the disease or injury being treated or prevented and the route of administration. The determination of suitable dosages and treatment regimens may be readily accomplished based upon information generally known in the art.

Embodiments of the invention also include methods of evaluating suitability of a subject for participating in a stem cell procedure. In such embodiments, the methods include obtaining a microRNA transcriptional phenotype for a sample from the subject, e.g., using a protocol such as described above, and then comparing the obtained microRNA transcriptional phenotype to a control, such as a healthy reference or a damaged reference, to evaluate suitability of the subject for the stem cell procedure. If the stem cell composition matches a healthy reference, the method may include determining that the subject is suitable for the stem cell procedure. If the stem cell composition matches a damaged reference, the method may include determining that the subject is unsuitable for the stem cell procedure.

The subject may be the recipient and/or donor of a stem cell composition for a stem cell procedure. For example, the methods may include obtaining a microRNA transcriptional phenotype for a potential donor of a stem cell composition in an allograph stem cell therapeutic procedure. If the microRNA transcriptional phenotype matches a healthy reference, the methods may include determining that the potential donor is suitable as a donor of a stem cell composition for the stem cell procedure. If the microRNA transcriptional phenotype matches a damaged reference, the methods may include determining that the potential donor is unsuitable as a donor of a stem cell composition for the stem cell procedure. In such instances, the method may further include evaluating one or more additional potential donors to identify a donor suitable for use in the stem cell procedure.

The subject may also be the patient, e.g., where the stem cell composition is to be employed in an autograph procedure or where the stem cell composition is obtained from a donor to be employed in an allograph procedure. For example, the methods may include obtaining a microRNA transcriptional phenotype for a potential recipient of a stem cell composition in an allograph stem cell therapeutic procedure. If the microRNA transcriptional phenotype matches a healthy reference, the methods may include determining that the potential recipient is suitable as a recipient of a stem cell composition for the stem cell procedure. If the microRNA transcriptional phenotype matches a damaged reference, the methods may include determining that the potential recipient is unsuitable as a recipient of a stem cell composition for the stem cell procedure. In such instances, the method may further include identifying an alternative therapeutic regimen for the potential recipient.

The above methods find use in a variety of different applications. Certain applications are now reviewed in the following Utility section.

### UTILITY

The subject methods and compound compositions find use in a variety of applications, including diagnostic, therapeutic and research applications.

In some instances, practice of subject methods results in treatment of a subject for a disease condition. By treatment is meant at least an amelioration of one or more symptoms associated with the disease condition afflicting the subject, where amelioration is used in a broad sense to refer to at least a reduction in the magnitude of a parameter, e.g., symptom, associated with the pathological condition being treated. As such, treatment also includes situations where the pathological condition, or at least symptoms associated therewith, are completely inhibited, e.g., prevented from happening, or stopped, e.g., terminated, such that the subject no longer suffers from the pathological condition, or at least the symptoms that characterize the pathological condition. Treatment may also manifest in the form of a modulation of a surrogate marker of the disease condition, e.g., as described above.

In certain embodiments, the stem cells and compositions comprising the same that are determined to be undamaged by methods of the invention are used to treat a clinically obvious injury or disease in a patient. In other embodiments, they are used prophylactically to prevent sub-clinically non-obvious injury or disease. In addition, in certain embodiments, they are used autologously to treat a patient from which the stem cells were obtain, while in other embodiments, they are used allogeneically to treat a patient other than the donor from which the stem cells were obtained. In one embodiment, they are used to treat a patient of the same species, while in another embodiment, they are used to treat a patient of a difference species, i.e., xenogeneic.

In certain embodiments, the stem cells and related compositions that are determined to be undamaged by methods of the invention are used to treat a variety of different diseases, including but not limited to inflammatory diseases, cardiovascular diseases, nervous system diseases, tumors, demyelinating diseases, digestive system diseases, endocrine system diseases, reproductive system diseases, hemic and lymphatic diseases, immunological diseases, mental disorders, musculoskeletal diseases, neuromuscular diseases, metabolic diseases, skin and connective tissue diseases, urological diseases, e.g., incontinence, erectile dysfunction, etc.

In various embodiments, the stem cells and related compositions determined to be undamaged by methods of the invention are used to treat a variety of different wounds, including but not limited to, abrasions, avulsions, blowing wounds, incised wounds, burns, contusions, puncture wounds, surgical wounds and subcutaneous wounds.

In some embodiments, the stem cells and related compositions determined to be undamaged by methods of the invention are used to treat or prevent a variety of injuries, including but not limited to, injuries to muscle, connective tissue (including tendon, ligament and cartilage), bone, lung tissue, blood vessels, nerve, liver, musculo-skeletal tissue or cardiac tissue. In some embodiments, the injury is a sports related injury, which includes but is not limited to contusions, myositis, strains, (including muscle and tendon strains), microtears, fractures (including avulsion fractures), dislocation, tear, sprains, stress fractures, bursitis, and articular cartilage injury.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some embodiments, the host is human.

### Kits & Systems

Also provided are kits and systems that find use in practicing embodiments of the methods, such as those described as described above. The term “system” as employed herein refers to a collection of two or more different active agents, present in a single or disparate composition, that are brought together for the purpose of practicing the subject methods. The term kit refers to a packaged active agent or agents. In some embodiments, the subject system or kit includes a dose of a subject compound (e.g., as described herein) and a dose of a second active agent (e.g., as described herein). The various kit components may be present in the containers, e.g., sterile containers, where the components may be present in the same or different containers.

In some instances, the kit may include a device for measuring the expression level of at least one miRNA in a sample, wherein said device comprises or consists of at least one probe or probe set for at least one miRNA of interest, e.g., as described above. In one embodiment, the device according to the present invention further comprises one or more probes or probe sets for one or more miRNAs of interest, e.g., as described above. In one embodiment said device comprises between 1 to 2 probes or probe sets per miRNA to be measured, such as 2 to 3 probes, for example 3 to 4 probes, such as 4 to 5 probes, for example 5 to 6 probes, such as 6 to 7 probes, for example 7 to 8 probes, such as 8 to 9 probes, for example 9 to 10 probes, such as 10 to 15 probes, for example 15 to 20 probes, such as 20 to 25 probes, for example 25 to 30 probes, such as 30 to 40 probes, for example 40 to 50 probes, such as 50 to 60 probes, for example 60 to 70 probes, such as 70 to 80 probes, for example 80 to 90 probes, such as 90 to 100 probes or probe sets per miRNA of the present invention to be measured. In another embodiment, said device has of a total of 1 probe or probe set for at least one miRNA to be measured, such as 2 probes, for example 3 probes, such as 4 probes, for example 5 probes, such as 6 probes, for example 7 probes, such as 8 probes, for example 9 probes, such as 10 probes, for example 11 probes, such as 12 probes, for example 13 probes, such as 14 probes, for example 15 probes, such as 16 probes, for example 17 probes, such as 18 probes, for example 19 probes, such as 20 probes, for example 21 probes, such as 22 probes, for example 23 probes, such as 24 probes, for example 25 probes, such as 26 probes, for example 27 probes, such as 28 probes, for example 29 probes, such as 30 probes, for example 31 probes, such as 32 probes, for example 33 probes, such as 34 probes, for example 35 probes, such as 36 probes, for example 37 probes, such as 38 probes, for example 39 probes, such as 40 probes, for example 41 probes, such as 42 probes, for example 43 probes, such as 44 probes, for example 45 probes, such as 46 probes, for example 47 probes, such as 48 probes, for example 49 probes, such as 50 probes or probe sets for at least one miRNA of the present invention to be measured. It follows, that there may be one probe specific to a miRNA to be measured, or more than one probe specific to a miRNA to be measured—which may be called a probe set. In one embodiment, the device comprises 1 probe per miRNA to be measured, in another embodiment, said device comprises 2 probes, such as 3 probes, for example 4 probes, such as 5 probes, for example 6 probes, such as 7 probes, for example 8 probes, such as 9 probes, for example 10 probes, such as 11 probes, for example 12 probes, such as 13 probes, for example 14 probes, such as 15 probes per miRNA to be measured or analyzed. In one embodiment, the device may be a microarray chip; a QPCR Micro Fluidic Card; or may comprise QPCR tubes, QPCR tubes in a strip or a QPCR plate, comprising one or more probes for at least one miRNA and identified herein. The probes may be comprised on a solid support, on at least one bead, or in a liquid reagent comprised in a tube.

In addition to the above-mentioned components, kits may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXPERIMENTAL

### I. Dyslipidemia is a Major Factor in Stem Cell Damage Induced by Uncontrolled Long-Term Type 2 Diabetes and Obesity in the Rat, as Suggested by the Effects on Stem Cell Culture (72)

### A. Introduction

Stem cell implantation into various types of damaged tissues has emerged as a promising therapy for several human urological disorders based on solid experimental evidence in animal models, and specifically in sexual medicine for erectile dysfunction (1,2) and Peyronie's disease (3,4). These studies have translated to therapy the extensive biological research showing the ability of stem cells to proliferate indefinitely and differentiate into the desired cell lines intended to replace the lost or damaged cells, or alternatively assuming that they may favorably change the damaged tissue composition through paracrine or juxtacrine effects. The main premise for their efficacy and safety is the belief, or rather the lack of contrary evidence, that the “immortal” stem cells are essentially resistant in their tissues of origin, and later in their sites of implantation, to the chronic noxious conditions and endogenous factors in many diseases, and even some physiological processes that damage the differentiated cells. This assumption extends to the tissue repair elicited by endogenous (rather than implanted) local or recruited stem cells, such as in the penile corpora cavernosa (5,6) or tunica albuginea (7,8), where the potential damage to stem cells by the tissue milieu affected by the disease has not been studied.

Sixteen human clinical trials using implanted stem cells are registered for the treatment of erectile dysfunction in www.clinicaltrials.gov, but only 3 of them are in the USA with none in academic research institutions or hospitals, and two trials are listed as ongoing for Peyronie's disease, but only one in the USA in a non-academic research institution. Proof of efficacy evaluated by the U.S. Federal Government is still pending. In addition, multiple direct therapeutic applications of stem cell implantation in humans for these and other conditions are offered, as well as ongoing small research studies not registered in the clinicaltrials.gov site, generally with little or no information on previous supporting research or adequacy of study design. The common denominator in clinical applications or studies is the use of autograft (autologous) implants, from the same patients that are being treated, mainly because of allogenic or syngeneic tissue rejection concerns when using stem cell banks. However, little is known regarding the efficacy and long-term safety of these procedures.

A main issue for all experimental stem cell therapy research in animal models driving the successive human use is rarely, if ever, discussed. This is that most or all studies are based on syngeneic, not on autologous, implantation, thus ignoring the various noxious systemic milieus that may affect cells in situ prior to their use in humans as autografts. In other words, the stem cells are obtained mostly from healthy young animals, i.e., not having experienced the impact of chronic conditions such as aging, cancer, vascular disease, heavy smoking, severe obesity, diabetes, and others, and implanted as a sort of cell bank into the host animals. So far, the main novel related research on stem cell damage is purely on biological and autocrine, rather than exocrine, processes: the stem cell senescence (9,10), without understanding more precisely the host aging factors that trigger or accelerate it and present potential risks for their use in therapy.

Our previous study (11) defined the in situ impairment by a systemic or local noxious milieu in the tissue of origin, particularly long-term uncontrolled-type 2 diabetes (UC-T2D) and obesity in a rat model, and showed that in addition to their phenotype imprinting, these stem cells had lost the capacity to exert tissue and functional repair when implanted into another tissue damaged by the same environment in either syngenic or autologous hosts. There are some reports describing deleterious effects of the T2D milieu and its associated oxidative stress on the microenvironment impairing stem cells, inducing apoptosis, interfering with wound healing capacity, and other effects (e.g., 12-15), as well as obesity per se or associated with diabetes on the number of stem cells and progenitor cells, their lifespan, and other effects (e.g., 16-18). The impairment of stem cells was ascribed in some cases to dyslipidemia (e.g., 19, 20). Two very recent reviews referred to these recent papers and warned on potential effects on the therapeutic use of stem cells by the systemic and tissue milieu of metabolic syndrome and T2D (21) and obesity (22), even if the discussed approaches and mechanisms are rather different from the current study.

Specifically, we had shown (11) that muscle derived stem cells (MDSC) isolated from the skeletal muscle of aged male obese Zucker (OZ) rats with long-term UC-T2D and which we refer to as Late Diabetic MDSC (LD-MDSC) from animals suffering morbid obesity and corporal veno-occlusive dysfunction (CVOD), when implanted as allografts into the penile corpora cavernosa of these same type of rats, failed to repair either CVOD or the underlying corporal histopathology. Moreover, the LD-MDSC had an abnormal pro-fibrotic/inflammatory phenotype evidenced by their gene expression global transcriptional signature (gene-GTS). In contrast, MDSC obtained from very young rats with early UC-T2D which we refer to as Early Diabetes MDSC (ED-MDSC), were able as expected to repair both penile corporal tissue and erectile function (11). This was similar to what we found with MDSC obtained from a non-diabetic rat strain for the therapy of erectile dysfunction in other animal models for risk factors, (23,24), and also with MDSC from normal mice but implanted into a T2D mouse model of limb ischemia (25).

In the current study we aimed to: 1) identify whether dyslipidemia or hyperglycemia is the main factor in the in vivo UC-T2D/obesity milieu that may cause stem cell damage, and get an insight into possible mechanisms mainly by subjecting in vitro the rat ED-MDSC to short incubations with the hyperglycemic and dyslipidemic serum of aged OZ rats, some specific dyslipidemic factors, and/or high glucose; 2) determine whether these agents affect fat infiltration, apoptosis, cell proliferation, protein expression, and the transcriptional imprinting of gene-GTS; c) verify whether microRNA (miR)-GTSs, are more sensitive than gene-GTSs in differentiating in vivo damaged (LD-MDSC) from normal (ED-MDSC) stem cells, and in identifying the noxious factors and providing an initial approach to their mechanism of action.

miRs are of high relevance, because they regulate protein translation, and transcription indirectly, and are widely accepted novel regulators and potential early biomarkers for various processes. This includes alterations of stem cell differentiation, cell cycle, apoptosis, and other types of damage (e.g., 26-29), diabetes, obesity and dyslipidemia (e.g., 30-32), and inflammation, and fibrosis (e.g., 33, 34), to name just a few. There are some recent publications on miRs in erectile dysfunction, (e.g., 35, 36), but none on Peyronie's disease.

### B. Materials & Methods

1. Rat model, MDSC and serum isolation. Obese Zucker (OZ) rats (CrI:ZUC-Lepr^{la}, catalog 185), and their Lean (LZ) counterparts (+/?, catalog 186) were obtained from Charles River (Wilmington, Mass.), treated according to the ‘Principles of laboratory animal care’ (The National Institutes of Health) with an Institutional Animal Care and Use Committee-approved protocol, and used for MDSC and serum isolation. The OZ rats are a model of metabolic syndrome starting to evolve at around 3-4 months into frank T2D, reaching a moderate hyperglycemia and morbid obesity which do not manifest in the LZ rats, and that is associated with erectile dysfunction, diabetic nephropathy and arteriosclerosis (11, 50-52).

The MDSC were initially described by J. Huard's group and extensively characterized by them (see e.g., 37-40), as well as by us (11, 23-25, 41-43). The ED-MDSC were obtained from the gastrocnemius of 2-3 male OZ rats at 12 weeks of age, when they are only mildly hyperglycemic and overweight, as compared to their lean non-diabetic counterpart (LZ) rats (11). In turn, LD-MDSC were from aged male OZ rats at 32 weeks of age, already severely obese and dyslipidemic but with only moderate hyperglycemia (see results), and having T2D/obesity-related CVOD. Sca-1 selected MDSC were isolated as described previously (11, 23-25, 41-43). The latter is the cell population containing MDSC. Cells were replicated on regular culture flasks (no coating) and used in the 10th-15th passage, since the mouse counterparts have been maintained in our laboratory for at least 40 passages with the same, or even increasing, growth rate (25, 41). Flow cytometry was performed to show that these cultures were Sca 1+/CD34+/CD44+ cells and expressed the key stem cell gene Oct 4 (11, 41). Low (5.6 mM) or high (22.2 mM) glucose media were used respectively for ED-MDSC and LD-MDSC culture maintenance. Blood was withdrawn from the 32 week old OZ rats and from the age-matched LZ rats, and the sera (OZ serum and LZ serum, respectively) were obtained.

2. MDSC culture incubations. ED-MDSC were incubated at an initial 20% confluence for 4 days on 6-well or 12-well collagen-coated plates, or on 8-well-removable compartment collagen-coated plates, in DMEM/10% fetal calf serum/5.8 mM glucose. For testing the effects of dyslipidemia in duplicate wells, either aged OZ or LZ serum was added to 0.5-5%, or soluble forms of palmitic acid, Na salt, conjugated to albumin (PA) or cholesterol-beta methylcyclodextrin (CHOL), both from Sigma-Aldrich (St. Louis, Mo.), were added to a concentration of 0.5 and 1 mM or 50 and 100 mg/dl, respectively. Albumin and methyl-cyclodextrin were not used as vehicle controls because when they are added, they do not act as inert vehicles but are active agents in binding endogenous PA and cholesterol. Control wells contained no lipid additives. For testing the effects of hyperglycemia, glucose was added to a final concentration of 10-25 mM. Upon completion of treatment, the medium was discarded and cells were washed with PBS and subjected to fixation for histochemistry, or extracted fresh for protein for western blots, or for RNA for gene/miR-GTS, respectively.
3. Quantitative histochemistry. Cells on the 12 well plates were subjected to Oil Red O staining for detecting fat droplets (11, 23, 34), and cells on the 8-well removable-partition plates were used for TUNEL determinations for establishing the apoptotic index (36). Quantitative image analysis (QIA) (11, 23-25, 34) was performed by computerized densitometry on 100× or 200× magnification pictures, using multiple pictures in order to cover the wells with as many fields as necessary, followed by QIA of all fields.
4. Western blots (11, 23-25, 36) Protein extracts were subjected to western blot immuno-detection. The primary antibodies used were: a) calponin 1, mouse monoclonal (Santa Cruz Biotechnology, Inc. Santa Cruz, Calif.); b) α-smooth muscle actin (ASMA), mouse monoclonal (Sigma/Aldrich St Louis, Mo.); c) myostatin (GDF8), our mouse monoclonal against the myostatin carboxy-terminal 113 amino acids (44); d) proliferating cell nuclear antigen (PCNA) mouse monoclonal (Millipore, Billerica, Mass., USA), e) caspase 3, mouse monoclonal (Cell Signalling Technology, Danvers, Mass.); f) glyceraldehyde 3-phosphate dehydrogenase (GAPDH), mouse monoclonal (Millipore, Billerica, Mass., USA), as a reference housekeeping protein. and g) beta-actin, mouse monoclonal, (Santa Cruz Biotechnology) also as a housekeeping protein. Membranes were incubated with secondary anti-mouse IgG, horseradish peroxidase (HRP)-linked antibody (Cell Signaling Technology, Danvers, Mass.) or anti-rabbit IgG linked to HRP (Amersham GE, Pittsburgh, Pa.). Bands were visualized using luminol (SuperSignal West Pico; Chemiluminescent, Pierce, Rockford, Ill.). For negative controls, the primary antibody was omitted. Densitometric analysis was performed in certain cases as stated, correcting by the housekeeping proteins.
5. Global transcriptional signatures (GTS).

a) Gene-GTS (11, 36, 45-47). The alterations of mRNA levels on RNA isolated using the Qiagen RNeasy Micro Plus kit, and whose quality was determined by the Agilent 2100 Bioanalyzer, were estimated by DNA microarrays performed by the UCLA DNA microarray core, by the Affymetrix Rat Gene array for over 32,000 sequences, where various housekeeping genes allow the normalization between different samples. Only genes that were up- or downregulated by at least 2-fold were considered unless specifically detailed. The impact of the UC-T2D/obesity milieu on the LD-MDSC expressed as the ratios between the LD-MDSC values for each selected gene and the respective control ED-MDSC values previously reported (11), but here selected, reorganized in descending order, and used to tabulate all other treatment ratios in the in vitro experiments referred to the newly determined control ED-MDSC values.

b) miR-GTS (36, 45, 46). RNA was isolated from cells using the mirVana™ miRNA isolation kit (Ambion), and analysis was performed by Norgen Biotek Corporation (Thorold, ON, Canada) by next-generation sequencing for all miR transcripts listed in the Sanger miRBase Release 18.0. Treatment ratios, including the newly determined miR-GTS for ED-MDSC and LD-MDSC, were obtained as for gene-GTS, against the respective value for the ED-MDSC, and selected and tabulated as for gene-GTS (11). In this case, normalization was done by expressing per 10⁷ total raw reads, correcting for various levels of amplification. Also, in one case (Table 5, below) individual miR values were calculated for each specimen as per thousand of the total miRs in that specimen, which makes the comparison irrespective of the total raw reads, and ratios to the ED-MDSC were calculated as in the other cases. Both the gene-GTS and miR-GTS complete results are deposited in the GEO library.

6. Statistical analysis. When applicable, values are expressed as the mean±SEM. The normality distribution of the data was established using the Wilk-Shapiro test. Multiple comparisons were analyzed by single factor ANOVA, followed by post hoc comparisons with the Bonferroni multiple comparison test.

### C. Results

1. The Age Progression of Hyperglycemia and Dyslipidemia in the OZ and LZ Rats Shows that the LD-MDSC were Exposed In Vivo to a Noxious UC-T2D Milieu.

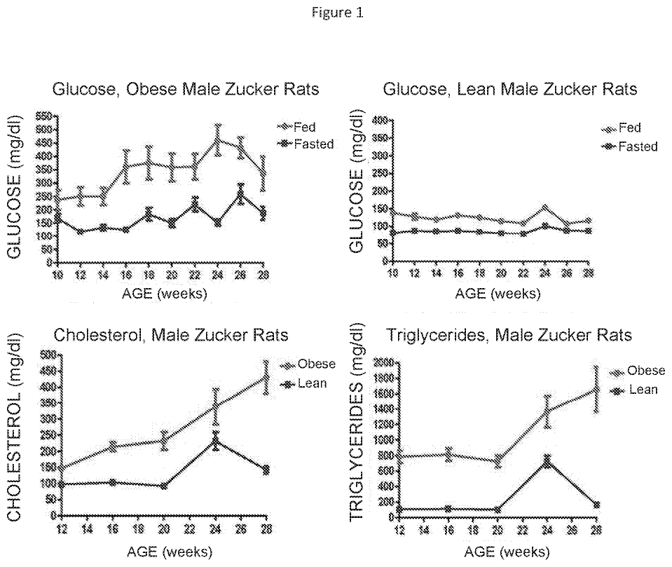

The ED-MDSC were isolated from 12 week old OZ rats, which when non-fasted had about 250 mg/dl glucose in the blood, whereas the age-matched LZ rats had 130 mg/dl (FIG. 1), showing that these stem cells from OZ rats were initially subjected to an early mild type 2 diabetes (UC-T2D), rather than just to insulin resistance, but for a relatively short period. In contrast, the LD-MDSC were isolated from OZ rats that had experienced a non-fasting hyperglycemia peak at 24 weeks of 460 mg/dl, decreasing to 338 mg/dl at 28 weeks, versus 120 mg/dl at the latter age in the LZ rats. At 32 weeks of age, when the rats were obtained, although glycemia had lowered to 202+/−8 mg/dl in the non-fasted OZ rats versus 104+/−4 in the LZ rats (not shown), this moderate or higher hyperglycemia had continued to exist in the LD-MDSC for at least 24 weeks.

Remarkably, these changes were accompanied by a steady increase of blood cholesterol from 150 mg/dl at 12 weeks of age to 420 mg/dl at 28 weeks in the non-fasted OZ rats, coupled to a consistent rise from 800 to 1,620 mg/dl in triglycerides, which in both cases presumably continued until 32 weeks of age (not measured). Therefore, the ED-MDSC were exposed short term to mild dyslipidemia, whereas the LD-MDSC were exposed long-term to very severe dyslipidemia.

2. Short-term in vitro exposure of ED-MDSC to dyslipidemia but not hyperglycemia induced stem cell damage, as evidenced by severe fat infiltration and apoptosis. The highly dyslipidemic conditions of the UC-T2D/obesity milieu were represented at 32 weeks by the aged OZ serum used for the experiments described below. In contrast, the LZ serum from the 32 week old rats was comparatively (but not strictly) normolipidemic, with 140 mg/dl for cholesterol and 160 mg/dl of triglycerides in the non fasted LZ rats at this peak. These OZ dyslipidemic changes were in agreement with severe obesity that reached at 32 weeks body weights of 745+/−26 g, vs only 494+/−15 g in the LZ rats (11).

We hypothesized that the aged OZ serum contains factors that would mimic the noxious in vivo milieu responsible for impairing the penile corporal tissue repair capacity of MDSC and their ability to restore erectile function and normal corporal histology (11). To test this, ED-MDSC, previously reported as “normal” based on their ability to provoke adequate tissue repair, were first incubated in monolayer cell culture for 4 days, with OZ serum added to 0.5%, 2.5%, and 5% final concentration in standard DMEM medium (containing 10% fetal calf serum), and compared with the LZ serum or with no addition. The DMEM-0.1% glucose (5.6 mM) medium was used in this and all subsequent experiments (unless stated) to mimic a normoglycemic milieu, thus excluding hyperglycemia as a confounding factor for these experiments.

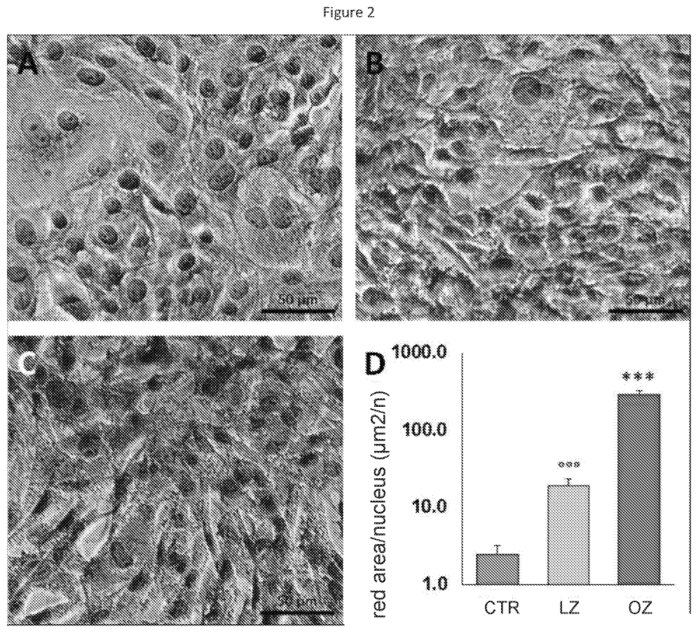

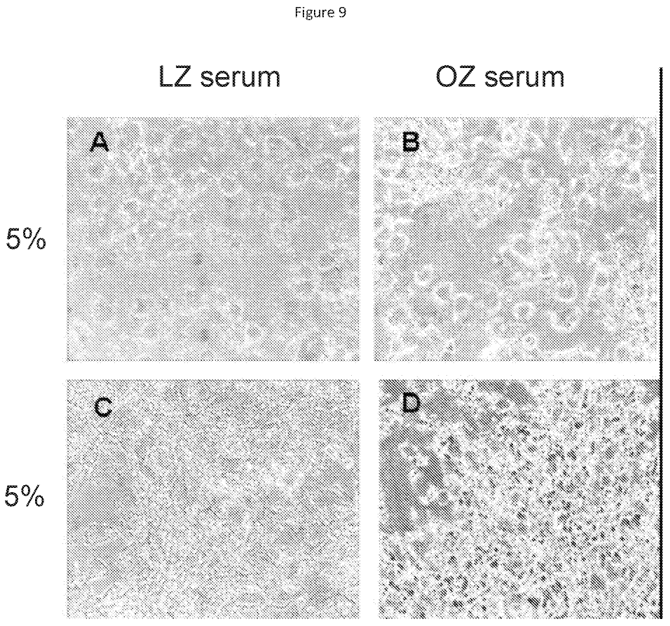

FIG. 9 B Supplement shows that even incubation with a very low 0.5% aged OZ serum for 4 days caused some fat infiltration in the ED-MDSC that would suggest stem cell damage, as denoted by Oil Red-O staining, and which was intensified at 2.5% (D), whereas this effect occurred to a much lower extent with the age-matched LZ serum at both concentrations (A and C). When aged OZ serum was increased to 5% (FIG. 2) the fat infiltration was very pronounced (C), but was essentially not observed following 5% LZ serum (B) and was not observed in the absence of rat serum addition (A). QIA confirmed the several-fold increase in stem cell fat infiltration for OZ serum versus LZ serum, expressed per stained nucleus (bar graphs; D, notice logarithmic Y axis).

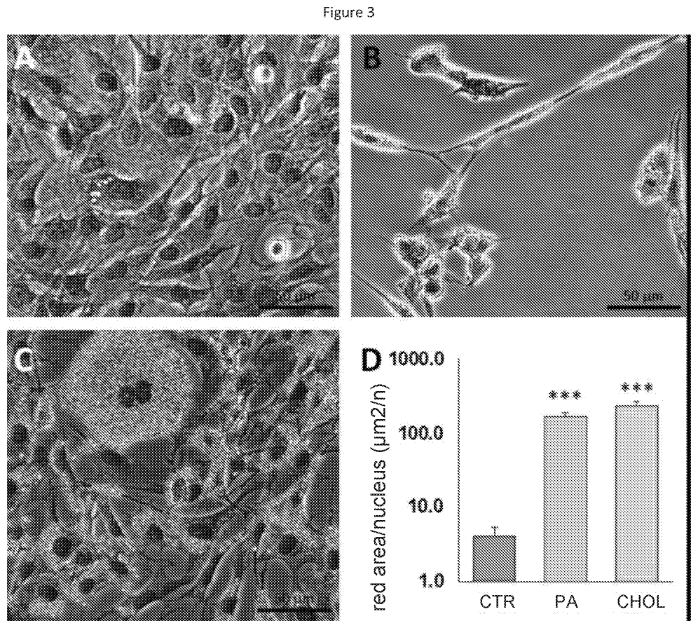

In order to determine whether key dyslipidemic factors result in the same fat infiltration in ED-MDSC as for the aged OZ serum, similar incubations were performed adding either a representative of free saturated fatty acids, i.e., solubilized palmitic acid (PA), at 0.5 (FIG. 3 B) and 1 mM, (not shown) or cholesterol, at 50 (C) and 100 (not shown) mg/dl, final concentrations, in comparison to no addition (A). The Oil Red O staining shows that there was lower fat infiltration induced by either PA or cholesterol, measured by QIA (D), as compared with the aged OZ serum effects shown in FIG. 2.

The aged OZ serum not only caused fat infiltration but also MDSC death, indicated by trypan blue staining (not shown). Despite the fact that all the aged OZ rats used for serum donation had very milky sera denoting high lipid concentrations, there were differences in the proportion of dead cells caused by each specific aged rat OZ serum added at 5%, except for 3 of them. One of these sera was selected for just a relatively mild cell death effect and was used for the experiments throughout the study. Dilution of the other deleterious sera to 1% showed a concentration dependence of trypan blue uptake, and thus a corresponding dependence of the cell-killing effect.

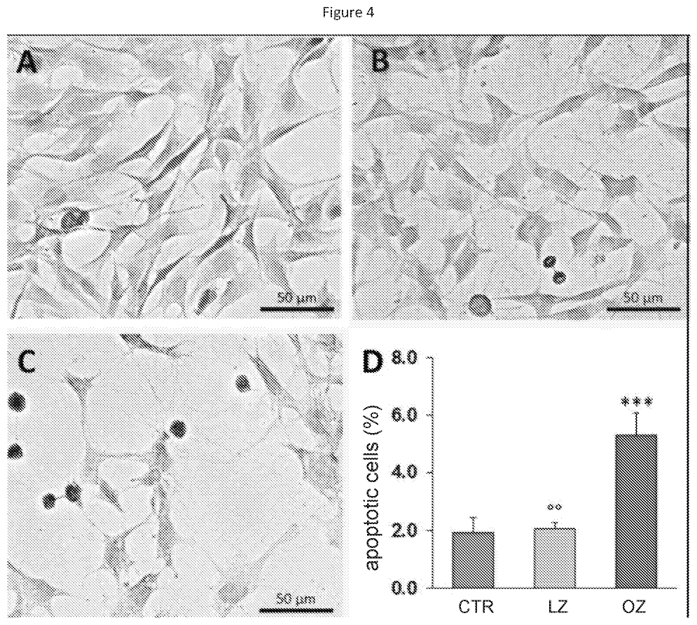

To determine whether the variable rates of stem cell death were at least in part caused by apoptosis, the effects of the selected aged OZ serum at 5% on the ED-MDSC (lower number of trypan blue positive cells) were assessed by the TUNEL reaction. FIG. 4 C shows that the 5% aged OZ serum used in this study caused MDSC apoptosis, but virtually none with LZ serum (B), and none in the absence of added serum (A). This was confirmed by QIA (bar graphs (D), shown in non-logarithmic scale. The apoptotic effects were apparently lower than the unspecific cell killing detected by the trypan blue staining, implying that there was in part some non-apoptotic cell death.

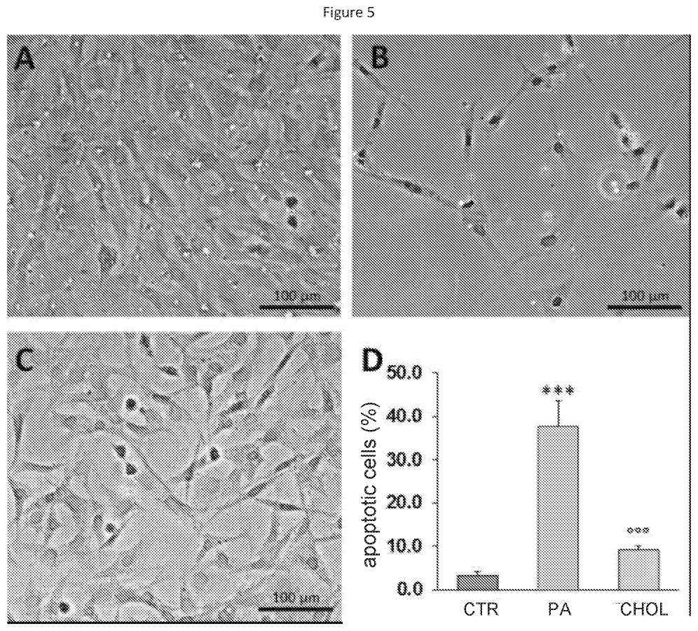

A much higher apoptosis induction, within a considerable general effect on cell death that reduced MDSC number, occurred with PA at 0.5 (FIG. 5 B) and 1 mM (not shown), and lower with cholesterol at 50 (C) and 100 mg/dl (not shown), with the expected absence of apoptosis without addition (A). QIA bar graphs (D) indicate this clearly in non-logarithmic scale for Y.

3. The fat infiltration and apoptosis was accompanied by reduced cell proliferation/differentiation, and myostatin over-expression.

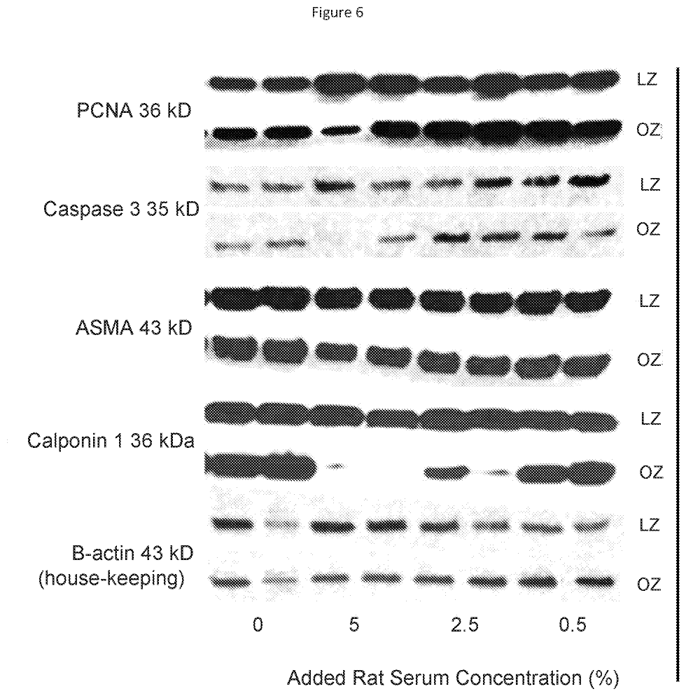

We then explored whether the aged OZ serum was able to induce some changes in protein expression affecting MDSC properties, as detected by western blot. FIG. 6 shows that ED-MDSC replication, represented by PCNA expression, was moderately reduced with aged OZ serum in comparison with its counterpart LZ serum, but only at 5%. Caspase 3, an indicator of mitochondrial apoptosis, was not increased contrary to what was expected, and was even mildly inhibited at 5% OZ serum, suggesting that the observed apoptosis resulted from other non-mitochondrial apoptotic pathways. ASMA, a smooth muscle cell and myofibroblast marker, was mildly reduced but not affected by higher concentrations. The spontaneous in vitro conversion of ED-MDSC into smooth muscle cells, a cell lineage that would be functionally desirable in penile corporal tissue repair, was more affected and severely reduced at 5%, as shown with calponin 1. The resulting increase in the ASMA/calponin 1 expression ratio would suggest a potentially deleterious spontaneous conversion into myofibroblasts.

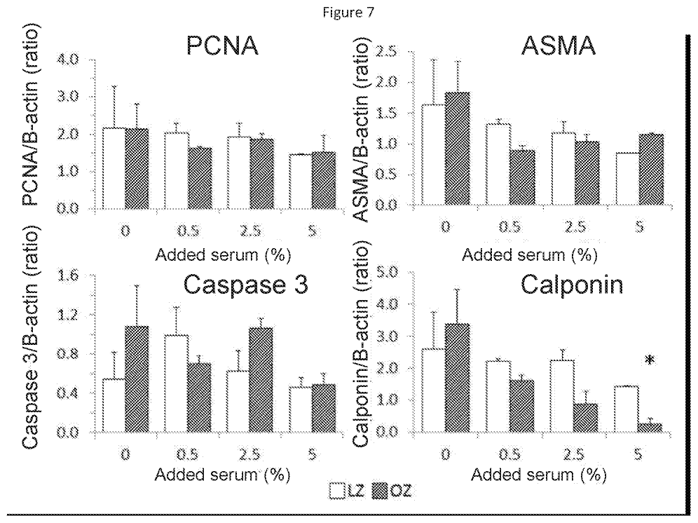

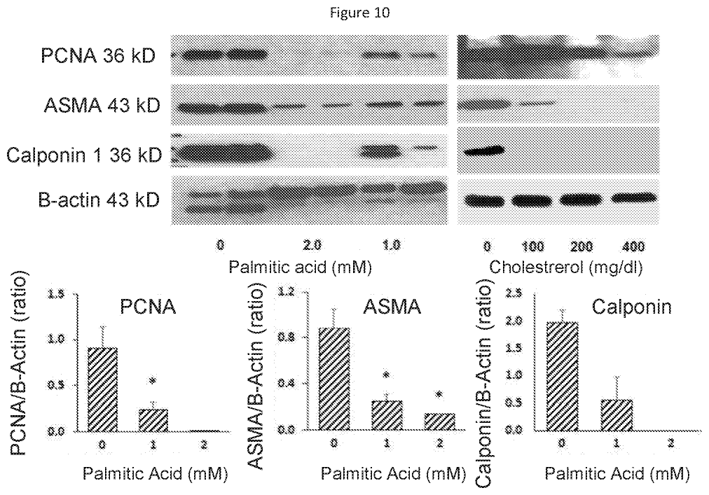

In turn Incubation with PA at 1 mM reduced PCNA, ASMA, and calponin 1 levels, and at 2 mM blocked considerably PCNA and calponin 1 expression, and presumably induced some palmitoylation evidenced by an increase in the larger beta-actin band (FIG. 10). As expected, cholesterol at all concentrations did not exert palmitoylation, but did block calponin-1, and also reduced ASMA. PCNA was lowered at only 200 and 400 mg/dl. FIG. 7 represents the quantitative densitometric determinations of band intensities only in the duplicate experiments, agreeing with the visual inspections, except that the PCNA decrease by serum was not significant. In turn, increasing concentrations of glucose failed in 1-week incubations of ED-MDSC to cause the changes induced by the aged OZ serum or PA (not shown).

We had reported that the repair-ineffective LD-MDSC, but not the repair-effective ED-MDSC, implanted into the corpora cavernosa of 24-week old OZ rats with CVOD, increased myostatin expression in the tissue, either from the MDSC themselves or from their interaction with the tissue (11). Therefore, we tested the possibility that the aged OZ serum could induce in vitro an effect similar to the one observed in vivo in the presence of the T2D/obesity milieu on the ED-MDSC. Myostatin is a lipofibrotic inducer and inhibitor of skeletal muscle mass made in this tissue, and a modulator of muscle/adipocyte differentiation, that we recently showed is also expressed in the corporal smooth muscle in the rat, and in the tunica albuginea myofibroblasts, and is weakly expressed in MDSC (11, 25, 48, 53, 54).

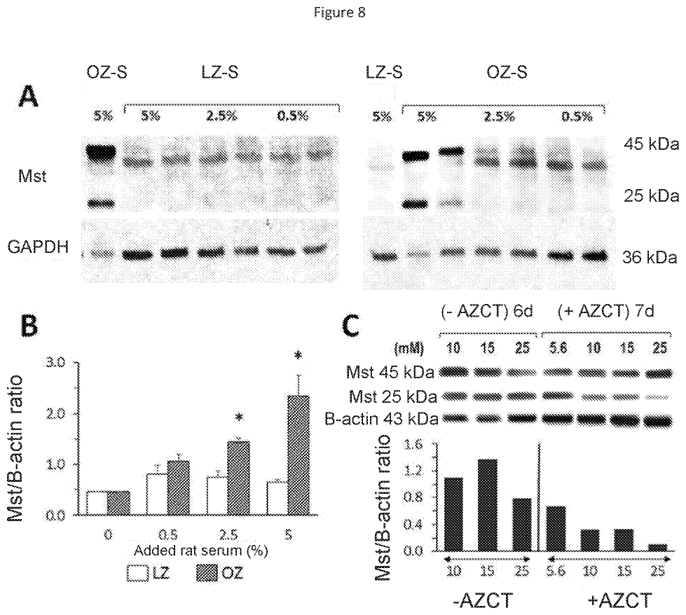

FIG. 8 shows that this was the case since there was a concentration-dependent increase of myostatin induced by aged OZ serum, in this case in the ED-MDSC themselves, not just in the MDSC/tissue interaction seen in vivo, and this did not occur with control age-matched LZ serum. Hyperglycemia per se was unable to induce over-expression of myostatin in the ED-MDSC when they were incubated for 1 week with increasing concentrations of glucose, either as such or after an azacytidine incubation to stimulate de-methylation and “stemness” (41). PA reduced myostatin expression (not shown).

4. The in vivo exposure of the LD-MDSC to the UC-T2D/obesity milieu induced a potential noxious imprint of their gene-GTS, which was partially reproduced by dyslipidemia in vitro, but not by hyperglycemia.

The induction of an abnormal gene GTS on ED-MDSC by 9 month exposure to the UC-T2D milieu is a landmark of stem cell damage and loss of tissue repair capacity, as previously reported (11). Therefore, we aimed to investigate here whether this could be replicated in vitro by short-term incubations under normal 0.1% (5.6 mM) glucose, with aged OZ serum, PA, or cholesterol, in comparison with 0.4% (22.4 mM) glucose alone. RNA was isolated and gene GTS were then obtained for the in vitro incubations. The respective basal data for the ED-MDSC controls and the LD-MDSC/ED-MDSC ratios in the in vivo series previously reported (11) are now presented in Tables 1 and 2 to allow for comparison with the new in vitro data. The respective ED-MDSC control values in the two series were used since the ED-MDSC were originated from different culture passages and the gene-GTS microarrays were done on different dates. In addition, values were standardized by similar RNA inputs, procedures, and internal housekeeping genes.

TABLE 1
Table 1

| | | | | In vitro effects of serum (S), | | | | |
|---|---|---|---|---|---|---|---|---|
| | | | | lipidemic factors (PA: palmitic acid; | | | | |
| | | In vivo T2D | | CHOL: cholesterol) and glucose (G) | | | | |
| In vivo LD/ED MDSC up-regulated | | effects | | (HG: high G; LG: low G) | | | | |
| genes (mRNAs) | | Basal | LD/ | Basal | OZS/ | CHOL/ | PA/ | HG/ |
| Gene ID | Gene Description | ED | ED | ED-2 | ED-2 | ED-2 | ED-2 | LG |
| Il1a | interleukin 1 alpha | 56 | 67.7 | 99 | 0.7 | 0.7 | 3.0 | 0.7 |
| Fgf7 | fibroblast qrowth factor 7 | 309 | 24.1 | 194 | 0.8 | 3.4 | 3.3 | 1.1 |
| Mt2A | metallothionein 2A | 758 | 20.8 | 251 | 0.5 | 3.3 | 1.2 | 0.6 |
| Cxcl1 | chemokine (C-X-C motif) | 476 | 17.2 | 577 | 0.6 | 2.6 | 6.4 | 0.9 |
| | liqand 1 | | | | | | | |
| Mmp9 | matrix metallopeptidase 9 | 176 | 15.0 | 173 | 0.7 | 1.9 | 0.9 | 0.9 |
| Angpt4 | angiopoietin 4 | 189 | 11.1 | 715 | 0.7 | 2.0 | 1.1 | 0.7 |
| Bdkrb1 | bradykinin receptor B1 | 88 | 9.8 | 74 | 0.8 | 2.7 | 1.6 | 1.2 |
| Ccr1 | chemokine (C-C motif) | 64 | 7.9 | 23 | 1.1 | 6.7 | 14.7 | 1.2 |
| | receptor 1 | | | | | | | |
| Itga2 | integrin, alpha 2 | 148 | 6.8 | 154 | 0.9 | 2.5 | 1.2 | 0.9 |
| Cxcl5 | chemokine (C-X-C motif) | 90 | 5.4 | 50 | 1.0 | 2.1 | 1.4 | 1.3 |
| | ligand 5 | | | | | | | |
| Il6 | interleukin 6 | 370 | 4.9 | 195 | 1.7 | 1.5 | 0.6 | 2.2 |
| Xdh | xanthine dehydrogenase | 400 | 4.5 | 619 | 1.0 | 2.4 | 0.9 | 1.3 |
| Wnt4 | wingless-type MMTV, | 306 | 4.5 | 842 | 0.7 | 1.5 | 0.3 | 0.8 |
| | member 4 | | | | | | | |
| Smad6 | SMAD family member 6 | 290 | 4.4 | 736 | 0.8 | 0.9 | 0.5 | 0.9 |
| Thbs2 | thrombospondin 2 | 746 | 4.1 | 5360 | 1.1 | 0.8 | 0.3 | 1.4 |
| Angpt1 | angiopoietin 1 | 70 | 4.0 | 180 | 1.7 | 0.7 | 1.0 | 2.1 |
| Cpt1a | carnitine palmitoyltransf | 412 | 3.5 | 472 | 1.7 | 1.7 | 2.0 | 1.0 |
| | 1a, liver | | | | | | | |
| Col15a1 | collagen, type XV, alpha 1 | 278 | 3.3 | 438 | 0.6 | 0.4 | 0.5 | 0.6 |
| Mmp3 | matrix metallopeptidase 3 | 638 | 3.2 | 2279 | 0.9 | 1.1 | 3.1 | 1.0 |
| Cxcl16 | chemokine (C-X-C motif) | 1407 | 3.1 | 2477 | 1.6 | 2.2 | 2.6 | 2.0 |
| | ligand 16 | | | | | | | |
| Bmp2 | bone morphogenetic | 153 | 2.9 | 257 | 0.9 | 1.8 | 3.5 | 1.2 |
| | protein 2 | | | | | | | |
| Mmp13 | matrix metallopeptidase 13 | 63 | 2.9 | 145 | 0.5 | 0.7 | 1.7 | 0.8 |
| Mmp23 | matrix metallopeptidase 23 | 712 | 2.9 | 2815 | 1.0 | 0.8 | 0.2 | 1.0 |
| Cd68 | Cd68 molecule | 109 | 2.6 | 109 | 1.1 | 3.0 | 1.4 | 1.0 |
| Tgfbr3 | transforming growth | 157 | 2.5 | 440 | 1.0 | 2.1 | 1.4 | 1.2 |
| | factor, beta rec, III | | | | | | | |
| Il7 | interleukin 7 | 79 | 2.4 | 98 | 0.7 | 1.2 | 1.9 | 0.9 |
| Cd274 | CD274 molecule | 291 | 2.3 | 69 | 1.2 | 1.8 | 2.2 | 1.4 |
| Mmp10 | matrix metallopeptidase 10 | 64 | 2.3 | 41 | 1.0 | 1.4 | 4.6 | 1.0 |
| Bcl2l11 | BCL2-like 11 (apoptosis | 207 | 2.3 | 136 | 0.9 | 2.1 | 1.6 | 1.2 |
| | facilitator) | | | | | | | |
| Myocd | myocardin | 2047 | 2.2 | 519 | 1.0 | 0.4 | 0.5 | 0.7 |
| Tnfrsf11b | tumor necrosis factor | 2323 | 2.1 | 1723 | 2.8 | 2.8 | 2.3 | 2.3 |
| | recept. family, 11b | | | | | | | |
| Pla2g2a | phospholipase A2, group | 51 | 2.0 | 268 | 1.0 | 4.2 | 2.7 | 1.2 |
| | IIA | | | | | | | |
| Mstn | myostatin | 56 | 1.5 | 721 | 1.5 | 0.1 | 1.4 | 1.2 |

The long-term in vivo exposure of MDSC to T2D in aged OZ rats (LD-MDSC) caused the transcriptional upregulation of many genes in comparison with the unexposed ED-MDSC, affecting inflammation, fibrosis, chemokines, and other noxious pathways, and most of these changes were replicated by the short term in vitro incubation with dyslipidemic factors, but not by hyperglycemia or aged OZ serum. The cell culture basal values for each gene in the global transcriptional signatures (GTS) for ED-MDSC were obtained separately for: a) the comparison with the GTS of LD-MDSC, and b) with all the in vitro incubation GTS comparisons, in order to compensate for different passage numbers and separate DNA microarray assays. The ratios for the in vivo exposure LD-MDSC/ED-MDSC, and for the different in vitro exposures of ED-MDSC vs the in vitro control ED-MDSC were obtained and highlighted in yellow when >2.0 or <0.5. The many up-regulated LD/ED genes are selected in this table for their relevance to stem cell damage related processes, and their IDs are highlighted in yellow only when one or more of the in vitro changes were in the same direction as the in vivo ones. Green highlighting indicates occasional opposite ratios.

Table 1 shows a selective comparison of the effects of the in viva exposure to the UC-T2D milieu, with only some previously reported (11), with the results of the 4 day in vitro exposure to aged serum, PA, cholesterol, or high glucose shown in the subsequent columns. The in vivo ratios between the LD-MDSC and the ED-MDSC, for genes that may be related to stem cell damage processes, are now sorted in decreasing order and restricted to the in vivo changes where mRNA levels were up-regulated in LD-MDSC versus ED-MDSC by >2.0 (represented by rounding up >1.95; highlighted in yellow). The respective in vitro changes that had the same direction (>1.95) are highlighted in yellow, or where opposite changes (<0.5); occurred, they are highlighted in green. Myostatin inclusion is the exception, since the LD/ED ratio was 1.5 in vivo, but is important in terms of its relevance to the miR-GTS changes presented in subsequent figures. The yellow gene IDs in the first column indicate that there was a similar direction (up-regulation) in any one of the in vitro changes and the few IDs without highlighting correspond to the absence of in vitro agreement with the in vivo values within the stated ranges. Of note for the discussion (not shown in the table), the LD-MDSC/ED-MDSC ratios for antioxidant enzymes in our gene-GTS were considerably elevated for glutathione peroxidase (Gpx3: 2.9) and superoxide dismutase (SOD3: 5.1), thus counteracting the only oxidant enzyme ratio that is elevated, the one for xanthine dehydrogenase (Xdh: 4.5), so that oxidative stress does not seem to act as a major factor in the in vivo MDSC damage.

Table 2 is structured similarly, but showing the down-regulation of the in vivo LD/ED ratios (i. e, <0.5), and compared to the respective <0.5 (yellow highlighting) or >2.0 (green) changes in the in vitro treatments.

TABLE 2
Table 2

| | | | | In vitro effects of serum (S), | | | | |
|---|---|---|---|---|---|---|---|---|
| | | | | lipidemic factors (PA: palmitic acid; | | | | |
| | | In vivo T2D | | CHOL: cholesterol) and glucose (G) | | | | |
| In vivo LD/ED MDSC down-regulated | | effects | | (HG: high G; LG: low G) | | | | |
| genes (mRNAs) | | Basal | LD/ | Basal | OZ/ | CHOL/ | PA/ | HG/ |
| Gene ID | Gene Description | ED | ED | ED-2 | ED-2 | ED-2 | ED-2 | LG |
| Ednra | endothelin receptor type A | 785 | 0.5 | 408 | 0.9 | 0.9 | 0.4 | 1.1 |
| Col12a1 | collagen, type XII, alpha 1 | 10193 | 0.5 | 10115 | 1.0 | 0.4 | 0.5 | 0.9 |
| Cdh3 | cadherin 3 | 2526 | 0.5 | 510 | 0.9 | 0.9 | 0.4 | 0.9 |
| Col6a1 | collagen, type VI, alpha 1 | 3359 | 0.5 | 3729 | 0.7 | 0.7 | 0.5 | 0.7 |
| Bmp6 | bone morphogenetic | 544 | 0.5 | 809 | 1.2 | 0.5 | 0.2 | 1.1 |
| | protein 6 | | | | | | | |
| Igfbp5 | insulin-like growth factor | 344 | 0.5 | 182 | 0.8 | 1.6 | 0.4 | 0.9 |
| | binding protein 5 | | | | | | | |
| Col3a1 | collagen, type III, alpha 1 | 3402 | 0.5 | 7135 | 0.7 | 0.6 | 0.4 | 0.8 |
| Tgfb2 | transforming growth | 5511 | 0.4 | 5305 | 1.0 | 0.5 | 0.4 | 0.9 |
| | factor, beta 2 | | | | | | | |
| Fzd2 | frizzled family receptor 2 | 2888 | 0.4 | 2526 | 0.8 | 0.8 | 0.3 | 0.9 |
| Myh10 | myosin, heavy chain 10, | 4476 | 0.4 | 1868 | 1.0 | 0.8 | 0.5 | 0.8 |
| | non-muscle | | | | | | | |
| Thbs4 | thrombospondin 4 | 1766 | 0.4 | 543 | 1.0 | 0.9 | 0.5 | 0.9 |
| Fads1 | fatty acid desaturase 1 | 6074 | 0.4 | 3411 | 0.6 | 0.5 | 0.6 | 0.7 |
| Cd200 | Cd200 molecule | 1692 | 0.4 | 356 | 2.1 | 0.9 | 1.2 | 2.8 |
| Myh1 | myosin, heavy polypeptide | 5196 | 0.3 | 1868 | 1.0 | 0.8 | 0.5 | 0.8 |
| | 1, sk muscle | | | | | | | |
| Pltp | phospholipid transfer | 1735 | 0.3 | 315 | 1.1 | 1.1 | 0.4 | 1.2 |
| | protein | | | | | | | |
| Cnnm2 | cyclin M2 | 1611 | 0.3 | 2564 | 1.0 | 0.5 | 0.7 | 1.2 |
| Casp12 | caspase 12 | 846 | 0.3 | 921 | 1.2 | 0.5 | 0.7 | 1.2 |
| Col11a1 | collagen, type XI, alpha 1 | 1658 | 0.3 | 2942 | 1.1 | 0.6 | 0.4 | 1.1 |
| Fads2 | fatty acid desaturase 2 | 4337 | 0.2 | 2839 | 0.6 | 0.3 | 0.4 | 0.7 |
| Casp4 | caspase 4, apoptosis-rel | 633 | 0.2 | 840 | 0.9 | 0.5 | 0.6 | 1.2 |
| | cysteine peptid | | | | | | | |
| Fzd8 | frizzled family receptor 8 | 2907 | 0.2 | 1713 | 0.9 | 0.5 | 0.4 | 1.1 |
| Adamtsl3 | ADAMTS-like 3 | 767 | 0.2 | 846 | 0.8 | 0.4 | 0.2 | 0.9 |
| Cxcl10 | chemokine (C-X-C motif) | 3152 | 0.2 | 370 | 1.1 | 0.9 | 3.7 | 0.9 |
| | ligand 10 | | | | | | | |
| Tnnc1 | troponin C type 1 (slow) | 1080 | 0.2 | 166 | 1.0 | 0.5 | 0.6 | 0.9 |
| Fgf1 | fibroblast growth factor 1 | 1828 | 0.2 | 2049 | 1.3 | 0.8 | 0.4 | 1.4 |
| Cmklrl | chemokine-like receptor 1 | 749 | 0.2 | 402 | 0.9 | 1.1 | 0.4 | 1.2 |
| Itga11 | integrin, alpha 11 | 7179 | 0.2 | 6784 | 0.9 | 0.7 | 0.4 | 0.9 |
| Itga4 | integrin, alpha 4 | 2243 | 0.1 | 843 | 1.7 | 0.5 | 0.5 | 0.9 |
| Fndc1 | fibronectin type III domain | 1772 | 0.1 | 1924 | 1.0 | 0.3 | 0.2 | 1.7 |
| | containing 1 | | | | | | | |
| Omd | osteomodulin | 477 | 0.1 | 116 | 1.3 | 0.5 | 0.2 | 2.6 |
| Itgbl1 | integrin, beta-like 1 | 2858 | 0.0 | 2129 | 1.5 | 0.9 | 0.4 | 1.7 |

The gene-GTS alteration caused in vivo by T2D or in vitro by dyslipidemia occurred also in opposite direction in a set of downregulated genes selected by their in vivo relevance. See Table 1 caption, but here only the LD-MDSC downregulated genes are selected with the same procedure as in Table 1.

Out of the >32,000 gene sequences (with gene ID) measured, only several hundred were up-regulated and we selected from them only 33 based on relevance for potential affected noxious pathways of the in vivo upregulated mRNAs. The majority of them (23) were changed in the same direction by either PA orcholesterol, and 6 by both agents, whereas remarkably only 1 was changed by aged OZ serum and 4 by hyperglycemia. Only 6 did not have any agreement in the in vitro results with the in vivo upregulated genes. Of note, the myostatin mRNA was uniformly increased in vivo in the LD-MDSC vs ED-MDSC and in vitro by serum, but only by a 1.5 factor. Cholesterol in vitro considerably downregulated myostatin. In turn, of the several hundred sequences that were downregulated we presented in Table 2 only 31 of the in vivo downregulated mRNAs that were changed in vitro in a similar direction, 20 of them by either PA or cholesterol, and 8 by both agents. Again, remarkably, none was with aged serum or with hyperglycemia, and only 2 did not show any in vitro correspondence.

Collectively this indicates that the dyslipidemic factors exerted in vitro a substantial number of changes in the gene-GTS in a similar direction as the imprinting of the LD-MDSC gene-GTS induced by the diabetic milieu in vivo, but high glucose in vitro was virtually inactive as shown for myostatin and other protein expression. This suggested that dyslipidemia rather than hyperglycemia was the main in vivo and in vitro factor in damaging the MDSC. However, the diabetic aged OZ serum was unable to imprint in vitro the ED-MDSC with the noxious in vivo gene-GTS phenotype seen in LD-MDSC, possibly due to the in vitro exposure being too short as to affect their gene-GTS in comparison with the several months operating in vivo.

5. The in vivo and in vitro gene-GTS changes induced by dyslipidemia were accompanied by parallel considerable changes in miR-GTS.

Therefore, we focused next on changes on a more sensitive and earlier GTS, that could precede and predict the final gene-GTS, namely potential alterations in the miR-GTS transcription. The RNA was this time isolated separately with a procedure tailored to miR recovery and quantification by next-generation sequencing. We had detected (above) over-expression of myostatin both as protein and as mRNA, and both in vivo in LD-MDSC, and in vitro in ED-MDSC with OZ-MDSC. Therefore, we have separated the miR-GTS data into two sets, as myostatin-related (Table 3) or myostatin-unrelated (Table 4) miRs, including all the in vitro treatment ratios vs the control values without addition, for each listed miR irrespective of the magnitude of their changes. This was done because of the putative relevance of myostatin protein overexpression by UC-T2D in vivo or by the aged OZ serum. The results have been compiled as down (<0.5) or up (>2.0) regulation of miR expression in the LD-MDSC vs ED-MDSC ratios, with the same rounding up or down as for the gene-GTS ratios, and presented together with the in vitro values.

TABLE 3
Table 3

Myostatin-

| related miRs | In vivo T2D | | In vitro effects of serum (S) and | | | | |
|---|---|---|---|---|---|---|---|
| changed | effects (OZ rat) | | lipidemic factors (PA: palmitic | | | | Reference |
| in amount | Basal value | Ratio to | acid; CHOL: cholesterol) | | | | numbers for |
| by T2D in | ED (per | ED-MDSC | Ratios to ED-MDSC | | | | myostatin- |
| LD vs ED | 10⁷reads) | LD/ED | OZS/ED | LZS/ED | PA/ED | CHOL/ED | related miRs |
| 23a-3p | 18,140 | 0.50 | 1.51 | 1.62 | 0.30 | 0.46 | 55 |
| 30e-5p | 6,244 | 0.42 | 0.71 | 0.77 | 0.54 | 0.58 | 56 |
| 27a-3p | 11,885 | 0.41 | 0.82 | 1.35 | 0.33 | 0.42 | 55, 58, 59, 61, |
| | | | | | | | 62, 67, See 60 |
| 181a-5p | 10,092 | 0.39 | 1.21 | 1.29 | 0.43 | 0.50 | 64 |
| 101a-3p | 2,549 | 0.38 | 1.62 | 2.26 | 0.51 | 0.57 | 57 |
| 199a-5p | 62,452 | 0.29 | 0.13 | 0.23 | 0.25 | 0.52 | 57, see 60 |
| 199a-3p | 20,968 | 0.25 | 0.15 | 0.37 | 0.33 | 0.63 | 57, see 60 |
| 29a-3p | 1,872 | 0.25 | 0.80 | 1.16 | 0.53 | 0.58 | 63 (as miR-29) |
| 214-3p | 4,056 | 0.22 | 0.09 | 0.15 | 0.25 | 0.67 | 64 |
| 21-5p | 547,830 | 0.16 | 0.32 | 0.31 | 0.26 | 0.41 | 65 |
| 101b-3p | 1,648 | 0.09 | 0.19 | 0.26 | 0.30 | 0.59 | 57 |
| 132-3p | 790 | 0.02 | 0.02 | 0.06 | 0.21 | 0.59 | 60 |

The long-term in vivo exposure of MDSC to the T2D diabetic milieu in the LD-MDSC, induced also a considerable change in the miR-GTS, and for this table specific miRs are selected by their relevance to myostatin, accompanied by many similar direction changes caused by treatment of MDSC in vitro with dyslipidemic serum and factors. See Tables 1 and 2 for captions, with individual, downregulated miRs selected here for their relevance to myostatin. The red highlighting indicates basal values >20,000 per 107 total raw reads so that every sample was normalized in this way. See Table 2 Supplement for the myostatin and stem cell related significance.

Table 3 shows that the in vivo T2D/obesity milieu: a) led to down-regulation of 12 miRs related to myostatin (with none upregulated), implying a subsequent up-regulation of myostatin mRNA/protein expression (as observed), since most miRs act as inhibitors of gene transcription/translation (the reference numbers 55-67 for the inter-relation of individual miRs with myostatin expression are indicated in the table); b) 6 of these miRs were also downregulated in vitro in the ED-MDSC by the aged OZ serum, and remarkably all by PA and 5 by cholesterol, 2 by all in vitro and in vivo (blue highlighting) and only in one case the result was opposite (upregulation) to the one in vivo; c) some of these changes were also seen with the aged LZ serum, but were less intensive; and d) the changes, in this case down-regulation, were more intense for the MDSC miR-GTS than for the MDSC gene-GTS. Based on the level of miR expression (red highlight), the observed down-regulation, its correlation with in vitro changes, and the relevance of each miR to myostatin and stem cell damage-related factors, miRs 21, 199a,b, 27a, 23a, and 181a, stand out.

TABLE 4
Table 4

| | | | In vitro effects of serum (S) and | | | |
|---|---|---|---|---|---|---|
| Other miRs | In vivo T2D | | lipidemic factors (PA: palmitic | | | |
| changed | effects (OZ rat) | | acid; CHOL: cholesterol) | | | |
| in amount | Basal value | Ratio to | Ratios to ED | | | |
| by T2D in | ED (per | ED | OZS/ | LZS/ | PA/ | CHOL/ |
| LD vs ED | 10⁷ reads) | LD/ED | ED | ED | ED | ED |
| 99a-5p | 30,750 | 2.65 | 1.75 | 2.25 | 0.29 | 0.32 |
| 25-3p | 6,350 | 2.20 | 1.90 | 5.17 | 2.07 | 1.53 |
| 10b-5p | 11,639 | 2.20 | 2.67 | 4.36 | 0.15 | 0.45 |
| 28-5p | 1,234 | 0.36 | 0.30 | 0.40 | 0.22 | 0.29 |
| 152-3p | 22,285 | 0.33 | 0.20 | 0.40 | 0.32 | 0.58 |
| 26a-5p | 58,443 | 0.32 | 0.79 | 1.57 | 0.34 | 0.66 |
| Let-7f-5p | 64,915 | 0.32 | 0.71 | 1.21 | 0.38 | 0.45 |
| 31a-5p | 2,868 | 0.29 | 0.17 | 0.35 | 0.49 | 0.65 |
| 196b-5p | 881 | 0.27 | 0.17 | 0.44 | 0.34 | 0.53 |
| 342-3p | 4,379 | 0.26 | 0.20 | 0.31 | 0.25 | 0.52 |
| 99b-5p | 507,430 | 0.25 | 0.47 | 0.52 | 0.27 | 0.45 |
| 148a-3p | 16,262 | 0.25 | 0.22 | 0.30 | 0.53 | 0.58 |
| 221-5p | 14,946 | 0.24 | 0.57 | 0.33 | 0.32 | 0.34 |
| let-7g-5p | 15,492 | 0.22 | 0.89 | 1.17 | 0.38 | 0.41 |
| 100-5p | 409,443 | 0.22 | 0.19 | 0.25 | 0.24 | 0.48 |
| 362-5p | 852 | 0.20 | 0.35 | 0.64 | 0.52 | 0.40 |
| 92a-3p | 12,454 | 0.17 | 1.26 | 0.91 | 0.83 | 0.47 |
| 148b-3p | 21,215 | 0.16 | 0.52 | 0.64 | 0.27 | 0.30 |
| 146a-5p | 14,087 | 0.14 | 0.04 | 0.02 | 0.86 | 0.49 |
| 196a-5p | 1,584 | 0.14 | 0.36 | 0.62 | 0.44 | 0.50 |
| Let-7i-5p | 68,254 | 0.12 | 0.44 | 0.66 | 0.36 | 0.66 |
| 10a-5p | 16,557 | 0.10 | 0.19 | 0.26 | 0.13 | 0.28 |
| 10b-3p | 1,643 | 0.10 | 0.39 | 0.53 | 0.30 | 0.59 |
| 212-5p | 1,026 | 0.08 | 0.02 | 0.07 | 0.35 | 0.76 |
| 224-5p | 1,368 | 0.06 | 0.20 | 0.31 | 0.76 | 0.75 |

The in vivo induced changes in individual myostatin-related miRs were also accompanied by changes in other individual miRs assumed to be unrelated to myostatin that were reflected as well on the in vitro dyslipidemic treatments. See Table 3 for the caption, but these miRs are not so far associated with myostatin. See Table 7 for their stem cell related significance.

Table 4 shows in turn, miRs unrelated to myostatin, 3 upregulated and 22 downregulated as a result of long-term in vivo exposure to the UC-T2D/obesity milieu, with all but 5 in the same direction by the aged OZ serum in vitro, all but 5 by PA and all but 12 by cholesterol. Again, the non-diabetic aged LZ serum exerted similar changes as the aged OZ serum, but much less intensive. This suggests that many of these miRs selected with the same criteria (except that they are unrelated to myostatin), may be relevant to the dyslipidemic effects but in this case through myostatin unrelated pathways, such as miR 99a, 152, 26a, 99b, 100, 148b, 10 a, etc.

Remarkably, when the changes listed in Tables 3 and 4 are expressed now in Table 5 as ratios between the % changes for each one of these miRs over the total expression for the overall listed miRs for each sample (instead of between normalized by raw values), most of the myostatin-related miRs (IDs highlighted in blue) selected from Table 3 preserve approximately the changes presented in Table 3. The same applies to all the other non-myostatin related miRs from Table 4 (non-highlighted IDs), placed there together.

TABLE 5

| | | | % total | % total | % total | % total | % total |
|---|---|---|---|---|---|---|---|
| | | | miR | miR | miR | miR | miR |
| | basal | % total | LD/ | OZS/ | LZS/ | PA/ | CHOL/ |
| | value | miR | ED-SC | ED-SC | ED-SC | ED-SC | ED-SC |
| miR ID | ED-SC | ED-SC | Ratio | Ratio | Ratio | Ratio | Ratio |
| rno-miR-21-5p | 730812 | 19.03 | 0.32 | 0.58 | 0.40 | 0.89 | 0.85 |
| rno-miR-99b-5p | 676574 | 17.62 | 0.48 | 0.87 | 0.67 | 0.93 | 0.94 |
| rno-miR-143-3p | 610702 | 15.90 | 3.02 | 1.34 | 1.78 | 0.75 | 1.09 |
| rno-miR-100-5p | 545925 | 14.21 | 0.41 | 0.35 | 0.32 | 0.80 | 0.98 |
| rno-miR-146b-5p | 140482 | 3.66 | 0.05 | 0.07 | 0.07 | 0.07 | 0.91 |
| rno-let-7i-5p | 91006 | 2.37 | 0.22 | 0.87 | 0.86 | 1.24 | 0.98 |
| rno-miR-199a-5p | 83061 | 2.16 | 0.54 | 0.24 | 0.30 | 0.85 | 1.07 |
| rno-miR-99a-5p | 40999 | 1.07 | 5.06 | 3.28 | 2.91 | 1.00 | 0.67 |
| rno-miR-148b-3p | 28286 | 0.74 | 0.30 | 0.97 | 0.90 | 0.93 | 0.62 |
| rno-miR-199a-3p | 27888 | 0.73 | 0.48 | 0.28 | 0.47 | 1.13 | 1.28 |
| rno-miR-23a-3p | 24126 | 0.63 | 0.95 | 2.51 | 2.10 | 1.02 | 0.93 |
| rno-miR-10a-5p | 22077 | 0.56 | 0.19 | 0.36 | 0.33 | 0.44 | 0.58 |
| rno-miR-148a-3p | 21863 | 0.57 | 0.47 | 0.42 | 0.39 | 1.59 | 1.79 |
| rno-let-7g-5p | 20657 | 0.54 | 0.43 | 1.66 | 1.52 | 1.31 | 0.85 |
| rno-miR-125a-5p | 20027 | 0.52 | 1.95 | 2.27 | 2.43 | 2.15 | 2.15 |
| rno-miR-221-5p | 19998 | 0.52 | 0.46 | 1.06 | 0.43 | 1.10 | 0.59 |
| rno-miR-146a-5p | 18773 | 0.49 | 0.27 | 0.08 | 0.03 | 2.96 | 0.99 |
| rno-miR-145-3p | 18234 | 0.47 | 2.67 | 1.30 | 1.63 | 0.67 | 1.29 |
| rno-miR-92a-3p | 16605 | 0.43 | 0.34 | 2.36 | 1.17 | 2.85 | 0.97 |
| rno-miR-27a-3p | 15854 | 0.41 | 0.78 | 1.54 | 1.74 | 1.12 | 0.87 |
| rno-miR-10b-5p | 15518 | 0.40 | 4.19 | 4.99 | 5.65 | 0.54 | 0.93 |
| rno-miR-181a-5p | 13422 | 0.35 | 0.75 | 2.27 | 1.65 | 1.48 | 1.04 |
| p-rno-miR-25-1, 2 | 8468 | 0.22 | 4.19 | 3.53 | 6.69 | 7.09 | 2.95 |
| rno-miR-214-3p | 5394 | 0.14 | 0.43 | 0.18 | 0.20 | 0.83 | 1.36 |
| rno-miR-342-3p | 5369 | 0.14 | 0.50 | 0.36 | 0.41 | 0.91 | 1.07 |
| rno-miR-212-5p | 4823 | 0.13 | 0.14 | 0.05 | 0.09 | 1.20 | 1.54 |
| rno-miR-31a-5p | 3824 | 0.10 | 0.54 | 0.49 | 0.45 | 1.71 | 1.34 |
| rno-miR-28-3p | 3189 | 0.08 | 2.03 | 0.76 | 0.84 | 1.66 | 1.68 |
| rno-miR-103-3p | 2924 | 0.08 | 0.46 | 0.99 | 0.85 | 1.46 | 0.97 |
| rno-miR-29a-3p | 2490 | 0.06 | 0.47 | 1.51 | 1.49 | 1.81 | 1.18 |
| rno-miR-101b-3p | 2191 | 0.06 | 0.19 | 0.71 | 0.69 | 1.04 | 1.21 |
| rno-miR-196a-5p | 2112 | 0.05 | 0.27 | 0.67 | 0.80 | 1.49 | 1.02 |
| rno-miR-143-5p | 1489 | 0.04 | 5.54 | 0.84 | 0.98 | 1.05 | 1.66 |
| rno-miR-224-5p | 1368 | 0.04 | 0.11 | 0.38 | 0.40 | 1.04 | 1.52 |
| rno-miR-196b-5p | 1175 | 0.03 | 0.51 | 0.32 | 0.56 | 1.16 | 1.08 |
| rno-miR-362-5p | 1135 | 0.03 | 0.36 | 0.65 | 0.83 | 1.79 | 0.80 |
| rno-miR-486 | 1091 | 0.03 | 2.75 | 18.11 | 7.84 | 2.34 | 1.08 |
| rno-miR-132-3p | 1051 | 0.03 | 0.05 | 0.04 | 0.07 | 0.72 | 1.21 |
| Rno-miR-20a-5p | 1006 | 0.03 | 0.31 | 2.05 | 1.81 | 2.39 | 1.05 |

The normalization of miR-GTS by expressing their individual values as a percent of the sum of total miR reads in each sample agrees with the selected results of most of the previously tabulated myostatin-related and -unrelated miRs. In order to determine how the relative proportion of miRs in each sample was affected by treatment, the current table was restricted by sorting in descending order the miRs whose basal values were >1,000 in the ED-MDSC, i.e., the 85 top expression miRs in the control, and then the individual miRs % were calculated in all samples for the ED-MDSC compiled miRs. The ratios of the miR % for each in vivo or in vitro treated sample over the ED-MDSC control was entered, and finally, only the miRs with LD %/ED % in vivo values >1.95 or <0.54 were selected. Yellow highlighting indicates changes within this range in the LD/ED reference ratio and in the in vitro incubations, whereas green highlighting shows opposite changes in vitro vs in vivo. The blue highlighting of the miR IDs defines myostatin-related miRs presented in Table 3, and the gray highlighting those miRs who were changed in the same direction, in all vivo, and in vitro treatments.

The comparison between percentages shows (highlighted in yellow) the considerable agreement between the direction of changes in vivo and in vitro, and the brown highlighting of IDs indicate that at least 4 in vitro ratios are in the same direction as in the in vivo situation. The only opposite directions are highlighted in green. This alternative normalization by % is independent of the total miR expression values for each sample and therefore is not subject to variability in initial RNA inputs or rounds of amplification during sequencing, thus indicating that the changes are specific for each miR and not resulting simply from a general down- or up-regulation of miRs in total.

The few differences between Tables 3 and 4 versus Table 5 may derive from the fact that Tables 3 and 4 give approximately the actual individual and total miR amounts. However, the latter is subject to variability in initial RNA inputs or rounds of amplification during sequencing.

### D. Discussion

Our previous in vivo data (11) had pioneered the demonstration that in the OZ rat the in vivo long-term exposure of stem cells from an early diabetic stage, our ED-MDSC, to an uncontrolled T2D/obesity milieu induced their failure to repair the tissue by replacing their lost corporal smooth muscle cells, the functional CVOD and its underlying corporal histopathology, and this damage was associated with the LD-MDSC imprinting with a noxious gene-GTS, and, upon their corporal tissue implantation, with the overproduction of myostatin, a pro-lipofibrotic protein that affects stem cell lineage, inhibits the generation of skeletal muscle mass, and is also present in the smooth muscle (48).

In the current work we have now extended these findings by pioneering an in vitro approach to reproduce some of these alterations and study their mechanism specifically by showing in short-term incubations of ED-MDSC: 1) the severe fat infiltration, apoptosis and non-apoptotic cell death, proliferation reduction (in the case of PA and CHOL), differentiation inhibition, and the overproduction of myostatin, induced specifically by the dyslipidemic serum, but none of these elicited by hyperglycemia; 2) the milder fat infiltration with the added dyslipidemic factors, water-soluble palmitate and cholesterol, but more intense apoptosis, particularly with PA, confirming the noxious role of dyslipidemia; 3) their association with changes in the gene-GTS, only some resembling the ones observed in vivo under the T2D/obesity milieu, but with changes in the miR-GTS that are representative of the in vivo observed miR-GTS; and 4) the significance of the miR-GTS for their potential use as a diagnostic tool to detect the stem cells damaged by their original in vivo exposure to the T2D/obesity milieu, in this case the LD-MDSC.

Our current experimental results agree with, and confirm, some of the assumptions from a recent review (21) condensed on its statement that “the environment (so-called niche) from which mesenchymal stem cells (MSC) are isolated may determine their usefulness”, particularly in relation to the review citations on the role of apoptosis and the reduction of proliferation in their deterioration induced by metabolic syndrome and T2D. However, our results do not seem to support the review's proposed main role of oxidative stress and reactive oxygen species (ROS) in stem cell damage, at least in or case in the LD-MDSC. Moreover, our evidence on dyslipidemia as a major factor in the stem cell damage is different from the stem cell senescence or autophagy concepts proposed there. In turn, our work has not focused on the putative (21) membrane derived vesicles role via AMK/mTOR on stem cell multipotency, but we do agree that the T2D/obesity induced release of cytokines (mainly interleukins and chemokines) may be worth to study.

We had previously discussed the implications of the gene-GTS alterations caused by a 9 month experience of the UC-T2D/obesity milieu in vivo (11), so it was now interesting to find that PA and to a lesser extent cholesterol, induced in vitro a much faster imprinting on many of those genes in just 4 days in the treated ED-MDSC. Again, hyperglycemia was ineffective in modifying gene-GTS, although based on its well-known effects through pathways such as AGE (advanced glycation end products) production we cannot discard that it may contribute to the dyslipidemia damage. However, although the added aged OZ serum induced some changes, they were not the expected ones that would mimic the in vivo modifications. We believe that the complex nature of the aged OZ serum composition, its dilution in vitro (added to only 5%) that reduces its dyslipidemic factor impact, and above all an insufficient time of contact, were potential reasons for blunting the effects of serum on the gene-GTS, in contrast to the UT-T2D/obesity milieu.

The discrepancy between some of the effects on the stem cells exerted by the T2D/obesity exposure in vivo and the aged OZ serum exposure in vitro, was compensated by the finding of a considerable and distinctive alteration of the miR-GTS found in the in vivo exposed LD-MDSC that was partially reproduced by the miR-GTS changes induced in vitro by the aged OZ serum. This makes sense if one considers that miRs are early inhibitors of gene transcription and translation, with miR-GTS changes necessarily preceding the resulting gene-GTS mRNA changes. Therefore, the miR-GTS is a more sensitive marker for the initial phenotype changes induced by a noxious factor than the gene-GTS. The fact that PA and to a much lesser extent, cholesterol, induced a much faster imprinting than the aged OZ serum of the gene-GTS in the ED-MDSC, and substantial changes in the miR-GTS, suggest that these factors are at least partially responsible for the dyslipidemic serum effects.

Of particular relevance, considering the observed in vivo UC-T2D/obesity-induced, and the in vitro aged OZ serum-induced upregulation of myostatin protein in MDSC and the lesser up-regulation of myostatin mRNA, is the in vivo and in vitro induced alterations of miR-GTS in both conditions for individual miRs related to myostatin. In fact, the selection of the miRs downregulated in vivo in the MDSC by the UC-T2D/obesity milieu in terms of their relationship to myostatin is validated in the bibliographic assessment presented in Table 6, by showing how each of these miRs are also related to potential stem cell damage processes.

TABLE 6

| | Total | | | | | | | | | | |
|---|---|---|---|---|---|---|---|---|---|---|---|
| | citations | Number of citations listed in Pub Med linking each miR-with keywords listed below | | | | | | | | | |
| miR- | Per miR- | | Stem | Stem Cell | | | | | Cell | | |
| or Mst | or Mst | Myostatin | Cells | differentiation | Inflammation | Fibrosis | Diabetes | Apoptosis | Proliferation | Fat | Dyslipidemia |
| 23a | 415 | 4 | 47 | 26 | 28 | 7 | 14 | 89 | 72 | 6 | 1 |
| 425 | 83 | 1 | 7 | 1 | 6 | 2 | 3 | 18 | 21 | 4 | 0 |
| 30e | 161 | 1 | 11 | 3 | 6 | 10 | 9 | 22 | 25 | 5 | 1 |
| 27a | 573 | 9 | 51 | 27 | 41 | 11 | 26 | 101 | 136 | 14 | 3 |
| 181a | 583 | 2 | 46 | 22 | 40 | 10 | 17 | 119 | 127 | 6 | 3 |
| 101a, b | 417 | 1 | 30 | 7 | 22 | 11 | 8 | 104 | 146 | 3 | 0 |
| as −101 | | | | | | | | | | | |
| 199a, b | 520 | 1 | 51 | 30 | 28 | 31 | 20 | 85 | 136 | 11 | 1 |
| 214 | 444 | 1 | 53 | 28 | 26 | 24 | 13 | 81 | 111 | 7 | 0 |
| 21 | 3,410 | 2 | 240 | 81 | 288 | 214 | 113 | 784 | 714 | 33 | 10 |
| 132 | 466 | 1 | 32 | 12 | 55 | 18 | 18 | 50 | 55 | 7 | 0 |
| Mst | 2,372 | 64 (miR) | 285 | 184 | 124 | 87 | 149 | 65 | 282 | 243 | 6 |

All myostatin-related miRs in Table 3 have relevance to stem cell damage-related processes, as judged by PubMed number of citations. Yellow highlighting: for the maximum number of citations within the selected processes. Blue highlighting: for the second number of citations. Gray highlighting: for most relevant miRs as judged by the combinations of miR changes, in vitro reproducibility, and stem cell-related relevance.
The observed down-regulation of miRs is in general associated with up-regulation of the respective gene, thus agreeing with the observed higher myostatin protein. Some of the selected miRs 21, 199a,b, 27a, 23a, and 181a, act either as inhibitors of myostatin expression or of downstream pathways (55-67), thus also acting as modulators of stem cell replication, and of damaging processes such as apoptosis, inflammation, fibrosis, etc., or are involved in dyslipidemia or UC-T2D pathways. The relevance of the in vivo and in vitro elicited changes in miR-GTS extend to processes affected by non-myostatin related miRs. (Table 7).

TABLE 7

| | Total | Number of citations listed in Pub Med linking each miR-with words listed below | | | | | | | | |
|---|---|---|---|---|---|---|---|---|---|---|
| miR- | citations | Stem | Stem Cell | | | | | Cell | | |
| ID | per miR- | Cells | Differ. | Inflammation | Fibrosis | Diabetes | Apoptosis | proliferation | Fat | DysLipidemia |
| 99a | 200 | 23 | 12 | 11 | 0 | 3 | 36 | 52 | 2 | 0 |
| 25 | 304 | 22 | 8 | 13 | 11 | 15 | 44 | 63 | 3 | 0 |
| 10b | 382 | 34 | 12 | 8 | 4 | 7 | 48 | 75 | 4 | 1 |
| 28 | 112 | 5 | 5 | 5 | 3 | 10 | 12 | 24 | 4 | 0 |
| 152 | 197 | 11 | 2 | 7 | 6 | 9 | 36 | 60 | 3 | 0 |
| 26a | 535 | 51 | 28 | 27 | 16 | 23 | 102 | 128 | 18 | 1 |
| Let-7f | 178 | 19 | 10 | 17 | 2 | 8 | 11 | 33 | 3 | 2 |
| 31a | 14 | 1 | 0 | 2 | 0 | 0 | 3 | 1 | 0 | 0 |
| 196b | 142 | 16 | 10 | 7 | 2 | 3 | 23 | 31 | 2 | 0 |
| 342 | 175 | 14 | 11 | 13 | 0 | 11 | 21 | 35 | 3 | 0 |
| 99b | 97 | 5 | 5 | 10 | 4 | 2 | 14 | 20 | 2 | 0 |
| 148a | 365 | 36 | 23 | 22 | 5 | 8 | 47 | 83 | 11 | 3 |
| 221 | 908 | 74 | 40 | 52 | 24 | 34 | 156 | 217 | 12 | 2 |
| Let-7g | 177 | 17 | 7 | 10 | 2 | 3 | 28 | 38 | 3 | 1 |
| 100 | 325 | 22 | 12 | 11 | 4 | 5 | 62 | 82 | 5 | 1 |
| 362 | 45 | 1 | 1 | 0 | 0 | 4 | 9 | 14 | 1 | 0 |
| 92a | 438 | 27 | 11 | 33 | 7 | 16 | 57 | 61 | 4 | 4 |
| 148b | 128 | 14 | 10 | 2 | 2 | 1 | 13 | 29 | 1 | 0 |
| 146a | 1,445 | 95 | 41 | 316 | 32 | 81 | 166 | 179 | 14 | 5 |
| 196a | 164 | 15 | 17 | 4 | 4 | 6 | 39 | 53 | 6 | 0 |
| Let-7i | 162 | 10 | 4 | 17 | 3 | 4 | 24 | 30 | 2 | 0 |
| 10a | 253 | 28 | 18 | 10 | 4 | 9 | 34 | 48 | 3 | 2 |
| 10b | 382 | 34 | 12 | 8 | 4 | 7 | 48 | 75 | 4 | 1 |
| 212 | 201 | 4 | 2 | 11 | 8 | 9 | 28 | 37 | 4 | 1 |
| 224 | 223 | 10 | 7 | 6 | 7 | 2 | 43 | 65 | 5 | 1 |

All myostatin-unrelated other miRs in Table 4 have relevance to stem cell damage-related processes, as judged by PubMed number of citations. See caption for Table 6.
Although their discussion exceeds the paper scope and length, miRs 99a, 100, Let 7a and 11a may be selected for further bibliographic analysis to explore their significance for stem cell damage through pathways other than myostatin.

We believe that the current work not only expands our previous observations (11), but it also pioneers an initial mechanistic approach by emphasizing dyslipidemia as the main noxious factor of MDSC damage, over hyperglycemia, and of an in vitro approach for a fast procedure to study these effects. The role of myostatin in this process is still unknown, but if its observed upregulation on the ED-MDSC by aged OZ serum is relevant to their fat infiltration and other damage, this would fit the well-known pro-adipogenic/obesogenic role of myostatin (68-70), and compound the stem cell damage with other noxious systemic changes.

This study expands and confirms our previous demonstration that long-term exposure of MDSC to the T2D2/obesity environment damages their tissue repair capacity and induces a noxious gene-GTS, by now showing that also the miR-GTS is considerably disturbed in vivo, particularly for miRs affecting myostatin expression, and that these alterations can be partially reproduced in vitro by short-term incubation of MDSC with highly dyslipidemic serum and dyslipidemic factors, which induce some similar miR-GTS changes, severe fat infiltration, apoptosis, overproduction of myostatin, and other noxious effects not seen with hyperglycemia alone. This allows in vitro mechanistic studies faster and easier to perform than in vivo. This implies, that both the therapeutic effects of implanted autologous MDSC in highly dyslipidemic T2D/obesity patients, their endogenous recruited MDSC cell repair, and even of other stem cells, may be compromised.

Kovanecz I., et al. (71) provides confirmation of the above findings in a female animal model and with female stem cells and confirms stem cell damage and the value of miR-GTS as biomarker of stem cell identity and damage.

### E. References

1. Matz E L, Terlecki R, Zhang Y, Jackson J, Atala A. Stem Cell Therapy for Erectile Dysfunction. Sex Med Rev. 2018 Apr. 6. pii: S2050-0521(18)30014-3. doi:10.1016/j.sxmr. 2017.12.008. [Epub ahead of print]
2. Reed-Maldonado A B, Lue T F. The Current Status of Stem-Cell Therapy in Erectile Dysfunction: A Review. World J Mens Health. 2016 December; 34(3):155-164.
3. Jiang H, Gao Q, Che X, Zhu L, Zhang Z, Chen Y, Dai Y. Inhibition of penile tunica albuginea myofibroblasts activity by adipose-derived stem cells, Exp Ther Med. 2017 November; 14(5):5149-5156.
4. Dellis A, Papatsoris A. Stem cell therapy for the treatment of Peyronie's disease. Expert Opin Biol Ther. 2017 April; 17(4):407-413.
5. Ruan Y, Lin G, Kang N, Tamaddon A, Zhou J, Wang B, Wang H S, Wang G, Banie L, Xin Z, Liu J, Lue T F. In Situ Activation and Preservation of Penile Progenitor Cells Using Icariside II in an Obesity-Associated Erectile Dysfunction Rat Model. Stem Cells Dev. 2018 Feb. 1; 27(3):207-215.
6. Vernet D, Heydarkhan S, Kovanecz I, Lue Y-H, Rajfer J, Gonzalez-Cadavid N F. Characterization of endogenous stem cells from the mouse penis that express an embryonic stem cell gene and undergo differentiation into several cell lineages. Am Urol Assoc Meet, Chicago, Ill., 2009 J Urol,
7. Xin Z C, Xu Y D, Lin G, Lue T F, Guo Y L. Recruiting endogenous stem cells: a novel therapeutic approach for erectile dysfunction. Asian J Androl. 2016 January-February; 18(1):10-5.
8. Vernet D, Nolazco G, Cantini L, Magee T R, Qian A, Rajfer J, Gonzalez-Cadavid N F. Evidence that osteogenic progenitor cells in the human tunica albuginea mayoriginate from stem cells: implications for peyronie disease. Biol Reprod. 2005 December; 73(6):1199-210.
9. Lidzbarsky G, Gutman D, Shekhidem H A, Sharvit L, Atzmon G. Genomic Instabilities, Cellular Senescence, and Aging: In Vitro, In Vivo and Aging-Like Human Syndromes. Front Med (Lausanne). 2018 Apr. 17; 5:104. doi: 10.3389/fmed.2018.00104. eCollection 2018.
10. Oh J, Lee Y D, Wagers A J. Stem cell aging: mechanisms, regulators and therapeutic opportunities. Nat Med. 2014 August; 20(8):870-80.
11. Kovanecz I, Vernet D, Masouminia M, Gelfand R, Loni L, Aboagye J, Tsao J, Rajfer J, Gonzalez-Cadavid N F. Implanted Muscle derived stem cells Ameliorate Erectile Dysfunction in a Rat Model of Type 2 Diabetes, but Their Repair Capacity Is Impaired by Their Prior Exposure to the Diabetic Milieu. J Sex Med. 2016 May; 13(5):786-97.
12. Zhu M, He X, Wang X H, Qiu W, Xing W, Guo W, An T C, Ao L Q, Hu X T, Li Z, Liu X P, Xiao N, Yu J, Huang H, Xu X. Complement C5a induces mesenchymal stem cell apoptosis during the progression of chronic diabetic complications. Diabetologia. 2017 September; 60(9):1822-1833. doi: 10.1007/s00125-017-4316-1. Epub 2017 Jun. 3.
13. Fadini G P, Ciciliot S, Albiero M. Concise Review: Perspectives and Clinical Implications of Bone Marrow and Circulating Stem Cell Defects in Diabetes. Stem Cells. 2017 January; 35(1):106-116.
14. Trinh N T, Yamashita T, Ohneda K, Kimura K, Salazar G T, Sato F, Ohneda O. Increased Expression of EGR-1 in Diabetic Human Adipose Tissue-Derived Mesenchymal Stem Cells Reduces Their Wound Healing Capacity. Stem Cells Dev. 2016 May 15; 25(10):760-73.
15. Mangialardi G, Spinetti G, Reni C, Madeddu P. Reactive oxygen species adversely impacts bone marrow microenvironment in diabetes. Antioxid Redox Signal. 2014 Oct. 10; 21(11):1620-33.
16. Xie G, Swiderska-Syn M, Jewell M L, Machado M V, Michelotti G A, Premont R T, Diehl A M. Loss of pericyte smoothened activity in mice with genetic deficiency of leptin. BMC Cell Biol. 2017 Apr. 20; 18(1):20. doi: 10.1186/s12860-017-0135-y.
17. Mitterberger M C, Mattesich M, Zwerschke W. Bariatric surgery and diet-induced long-term caloric restriction protect subcutaneous adipose-derived stromal/progenitor cells and prolong their life span in formerly obese humans. Exp Gerontol. 2014 August; 56:106-13.
18. Tsai T H (1), Chai H T, Sun C K, Yen C H, Leu S, Chen Y L, Chung S Y, Ko S F, Chang H W, Wu C J, Yip H K. Obesity suppresses circulating level and function of endothelial progenitor cells and heart function. J Transl Med. 2012 Jul. 2; 10:137. doi: 10.1186/1479-5876-10-137.
19. Dhulekar J, Simionescu A. Challenges in vascular tissue engineering for diabetic patients. Acta Biomater. 2018 Apr. 1; 70:25-34.
20. Chakravarthy H, Navitskaya S, O'Reilly S, Gallimore J, Mize H, Beli E, Wang Q, Kady N, Huang C, Blanchard G J, Grant M B, Busik J V. Role of Acid Sphingomyelinase in Shifting the Balance Between Pro-inflammatory and Reparative Bone Marrow Cells in Diabetic Retinopathy. Stem Cells. 2016 April; 34(4):972-83.
21. Kornicka K, Houston J, Marycz K. Dysfunction of Mesenchymal Stem Cells Isolated from Metabolic Syndrome and Type 2 Diabetic Patients as Result of Oxidative Stress and Autophagy may Limit Their Potential Therapeutic Use. Stem Cell Rev. 2018 June; 14(3):337-345.
22. Payab M, Goodarzi P, Heravani N F, Hadavandkhani M, Zarei Z, Falahzadeh K, Larijani B, Rahim F, Arjmand B. Stem Cell and Obesity: Current State and Future Perspective. Adv Exp Med Biol. 2018 Jun. 7. doi: 10.1007/5584_2018_227. [Epub ahead of print]
23. Kovanecz I, Rivera S, Nolazco G, Vernet D, Segura D, Gharib S, Rajfer J, Gonzalez-Cadavid N F. Separate or combined treatments with daily sildenafil, molsidomine, or muscle derived stem cells prevent erectile dysfunction in a rat model of cavernosal nerve damage. J Sex Med. 2012 November; 9(11):2814-26.
24. Nolazco G, Kovanecz I, Vernet D, Gelfand R A, Tsao J, Ferrini M G, Magee T, Rajfer J, Gonzalez-Cadavid N F. Effect of muscle derived stem cells on the restoration of corpora cavernosa smooth muscle and erectile function in the aged rat. BJU Int. 2008 May; 101(9):1156-64.
25. Tsao J, Kovanecz I, Awadalla N, Gelfand R, Sinha-Hikim I, White R A, Gonzalez-Cadavid N F. Muscle Derived Stem Cells Stimulate Muscle Myofiber Repair and Counteract Fat Infiltration in a Diabetic Mouse Model of Critical Limb Ischemia. J Stem Cell Res Ther. 2016 December; 6(12). pii: 370. doi:10.4172/2157-7633.1000370.
26. Mens M M J, Ghanbari M. Cell Cycle Regulation of Stem Cells by MicroRNAs. Stem Cell Rev. 2018 June; 14(3):309-322.
27. Hao J, Duan F F, Wang Y. MicroRNAs and RNA binding protein regulators of microRNAs in the control of pluripotency and reprogramming. Curr Opin Genet Dev. 2017 October; 46:95-103.
28. Pourrajab F, Vakili Zarch A, Hekmatimoghaddam S, Zare-Khormizi M R. The master switchers in the aging of cardiovascular system, reverse senescence by microRNA signatures; as highly conserved molecules. Prog Biophys Mol Biol. 2015 November; 119(2):111-28.
29. Li N, Long B, Han W, Yuan S, Wang K. microRNAs: important regulators of stem cells. Stem Cell Res Ther. 2017 May 11; 8(1):110. doi: 10.1186/s13287-017-0551-0.
30. Berry C, Lal M, Binukumar B K. Crosstalk Between the Unfolded Protein Response, MicroRNAs, and Insulin Signaling Pathways: In Search of Biomarkers for the Diagnosis and Treatment of Type 2 Diabetes. Front Endocrinol (Lausanne). 2018 May 2; 9:210. doi: 10.3389/fendo.2018.00210. eCollection 2018.
31. McGregor R A, Choi M S. microRNAs in the regulation of adipogenesis and obesity. Curr Mol Med. 2011 June; 11(4):304-16.
32. Desgagné V, Bouchard L, Guérin R. microRNAs in lipoprotein and lipid metabolism: from biological function to clinical application. Clin Chem Lab Med. 2017 May 1; 55(5):667-686.
33. Hirschberger S, Hinske L C, Kreth S. MiRNAs: dynamic regulators of immune cell functions in inflammation and cancer. Cancer Lett. 2018 May 22; 431:11-21.
34. Lv W, Fan F, Wang Y, Gonzalez-Fernandez E, Wang C, Yang L, Booz G W, Roman R J. Therapeutic potential of microRNAs for the treatment of renal fibrosis and CKD. Physiol Genomics. 2018 Jan. 1; 50(1):20-34.
35. Bai Y, Zhang L, Jiang Y, Ju J, Li G, Xu J, Jiang X, Zhang P, Lang L, Sadkovaya O, Glybochko P V, Zhang W, Yang B. Identification and Functional Verification of MicroRNAs in the Obese Rat with Erectile Dysfunction. Sex Med. 2017 December; 5(4):e261-e271.
36. Kovanecz I, Gelfand R, Masouminia M, Gharib S, Segura D, Vernet D, Rajfer J, Li D K, Kannan K, Gonzalez-Cadavid N F. Oral Bisphenol A (BPA) given to rats at moderate doses is associated with erectile dysfunction, cavernosal lipofibrosis and alterations of global gene transcription. Int J Impot Res. 2014 March-April; 26(2):67-75.
37. Lee J Y, Qu-Petersen Z, Cao B, Kimura S, Jankowski R, Cummins J, Usas A, Gates C, Robbins P, Wernig A, Huard J. Clonal isolation of muscle-derived cells capable of enhancing muscle regeneration and bone healing. J Cell Biol. 2000 Sep. 4; 150(5):1085-1100.
38. Jankowski R J, Haluszczak C, Trucco M, Huard J. Flow cytometric characterization of myogenic cell populations obtained via the preplate technique: potential for rapid isolation of muscle-derived stem cells. Hum Gene Ther. 2001 Apr. 10; 12 (6):619-28.
39. Deasy B M, Gharaibeh B M, Pollett J B, Jones M M, Lucas M A, et al. Long-term self-renewal of postnatal muscle-derived stem cells. Mol Biol Cell. 2005; 16:3323-3333.
40. Kwon D, Kim Y, Pruchnic R, Jankowski R, Usiene I, de Miguel F, Huard J,Chancellor M B. Periurethral cellular injection: comparison of muscle-derived progenitor cells and fibroblasts with regard to efficacy and tissue contractility in an animal model of stress urinary incontinence. Urology. 2006 August; 68(2):449-54.
41. Tsao J, Vernet D A, Gelfand R, Kovanecz I, Nolazco G, Bruhn K W, Gonzalez-Cadavid N F. Myostatin genetic inactivation inhibits myogenesis by muscle-derived stem cells in vitro but not when implanted in the mdx mouse muscle. Stem Cell Res Ther. 2013 Jan. 7; 4(1):4. doi: 10.1186/scrt152.
42. Wang J S, Kovanecz I, Vernet D, Nolazco G, Kopchok G E, Chow S L, White R A, Gonzalez-Cadavid N F. Effects of sildenafil and/or muscle derived stem cells on myocardial infarction. J Transl Med. 2012 Aug. 7; 10:159. doi: 10.1186/1479-5876-10-159.
43. Ho M H, Heydarkhan S, Vernet D, Kovanecz I, Ferrini M G, Bhatia N N, Gonzalez-Cadavid N F. Stimulating vaginal repair in rats through skeletal muscle-derived stem cells seeded on small intestinal submucosal scaffolds. Obstet Gynecol. 2009 August; 114(2 Pt 1):300-9.
44. Artaza J N, Bhasin S, Magee T R, Reisz-Porszasz S, Shen R, Groome N P, Meerasahib M F, Gonzalez-Cadavid N F. Myostatin inhibits myogenesis and promotes adipogenesis in C3H 10T(1/2) mesenchymal multipotent cells. Endocrinology. 2005 August; 146(8):3547-57
45. Gelfand R, Vernet D, Bruhn K W, Sarkissyan S, Heber D, Vadgama J V, Gonzalez-Cadavid N F. Long-term exposure of MCF-7 breast cancer cells to ethanol stimulates oncogenic features. Int J Oncol. 2017 January; 50 (1):49-65.
46. Gelfand R, Vernet D, Bruhn K, Vadgama J, Gonzalez-Cadavid N F. Long-term exposure of MCF-12A normal human breast epithelial cells to ethanol induces epithelial mesenchymal transition and oncogenic features. Int J Oncol. 2016 June; 48(6):2399-414.
47. Gelfand R A, Vernet D, Kovanecz I, Rajfer J, Gonzalez-Cadavid N F. The transcriptional signatures of cells from the human Peyronie's disease plaque and the ability of these cells to generate a plaque in a rat model suggest potential therapeutic targets. J Sex Med. 2015 February; 12(2):313-27.
48. Kovanecz I, Masouminia M, Gelfand R, Vernet D, Rajfer J, Gonzalez-Cadavid N F. Myostatin, a profibrotic factor and the main inhibitor of striated muscle mass, is present in the penile and vascular smooth muscle. Int J Impot Res. 2017 September; 29(5):194-201.
49. Kovanecz I, Ferrini M G, Vernet D, Nolazco G, Rajfer J, Gonzalez-Cadavid N F. Pioglitazone prevents corporal veno-occlusive dysfunction in a rat model of type 2 diabetes mellitus. BJU Int. 2006 July; 98(1):116-24.
50. Toblli J E, Cao G, Giani J F, Angerosa M, Dominici F P, Gonzalez-Cadavid N F. Antifibrotic effects of pioglitazone at low doses on the diabetic rat kidney are associated with the improvement of markers of cell turnover, tubular and endothelial integrity, and angiogenesis. Kidney Blood Press Res. 2011; 34(1):20-33.
51. Toblli J E, Ferrini M G, Cao G, Vernet D, Angerosa M, Gonzalez-Cadavid N F. Antifibrotic effects of pioglitazone on the kidney in a rat model of type 2 diabetes mellitus. Nephrol Dial Transplant. 2009 August; 24(8):2384-91.
52. Kovanecz I, Nolazco G, Ferrini M G, Toblli J E, Heydarkhan S, Vernet D, Rajfer J, Gonzalez-Cadavid N F. Early onset of fibrosis within the arterial media in a rat model of type 2 diabetes mellitus with erectile dysfunction. BJU Int. 2009 May; 103(10):1396-404.
53. Cantini L P, Ferrini M G, Vernet D, Magee T R, Qian A, Gelfand R A, Rajfer J, Gonzalez-Cadavid N F. Profibrotic role of myostatin in Peyronie's disease. J Sex Med. 2008 July; 5(7):1607-22.
54. Artaza J N, Singh R, Ferrini M G, Braga M, Tsao J, Gonzalez-Cadavid N F. Myostatin promotes a fibrotic phenotypic switch in multipotent C3H 10T1/2 cells without affecting their differentiation into myofibroblasts. J Endocrinol. 2008 February; 196(2):235-49.
55. Wang B, Zhang C, Zhang A, Cai H, Price S R, Wang X H. MicroRNA-23a and MicroRNA-27a Mimic Exercise by Ameliorating CKD-Induced Muscle Atrophy. J Am Soc Nephrol. 2017 September; 28(9):2631-2640.
56. Jia H, Zhao Y, Li T, Zhang Y, Zhu D. miR-30e is negatively regulated by myostatin in skeletal muscle and is functionally related to fiber-type composition. Acta Biochim Biophys Sin (Shanghai). 2017 May 1; 49(5):392-399.
57. Javed R, Jing L, Yang J, Li X, Cao J, Zhao S. miRNA transcriptome of hypertrophic skeletal muscle with overexpressed myostatin propeptide. Biomed Res Int. 2014; 2014:328935. doi: 10.1155/2014/328935. Epub 2014 Jul. 24.
58. McFarlane C, Vajjala A, Arigela H, Lokireddy S, Ge X, Bonala S, Manickam R, Kambadur R, Sharma M. Negative auto-regulation of myostatin expression is mediated by Smad3 and microRNA-27. PLoS One. 2014 Jan. 31; 9(1):e87687. doi: 10.1371/journal.pone. 0087687. eCollection 2014.
59. Chen X (1), Huang Z, Chen D, Yang T, Liu G. Role of microRNA-27a in myoblast differentiation. Cell Biol Int. 2014 February; 38(2):266-71.
60. Miretti S (1), Martignani E, Accornero P, Baratta M. Functional effect of mir-27b on myostatin expression: a relationship in Piedmontese cattle with double-muscled phenotype. BMC Genomics. 2013 Mar. 19; 14:194. doi: 10.1186/1471-2164-14-194.
61. Huang Z, Chen X, Yu B, He J, Chen D. MicroRNA-27a promotes myoblast proliferation by targeting myostatin. Biochem Biophys Res Commun. 2012 Jun. 29; 423(2):265-9.
62. Allen D L (1), Loh A S. Posttranscriptional mechanisms involving microRNA-27a and b contribute to fast-specific and glucocorticoid-mediated myostatin expression in skeletal muscle. Am J Physiol Cell Physiol. 2011 January; 300(1):C124-37.
63. Goodman C A, McNally R M, Hoffmann F M, Hornberger T A. Smad3 induces atrogin-1, inhibits mTOR and protein synthesis, and promotes muscle atrophy in vivo. Mol Endocrinol. 2013 November; 27(11):1946-57.
64. Zhu X, Chen D, Hu Y, Wu P, Wang K, Zhang J, Chu W, Zhang J. The microRNA signature in response to nutrient restriction and refeeding in skeletal muscle of Chinese perch (Siniperca chuatsi). Mar Biotechnol (NY). 2015 April; 17(2):180-9.
65. Hu S L, Chang A C, Huang C C, Tsai C H, Lin C C, Tang C H. Myostatin Promotes Interleukin-1β Expression in Rheumatoid Arthritis Synovial Fibroblasts through Inhibition of miR-21-5p. Front Immunol. 2017 Dec. 8; 8:1747. doi: 10.3389/fimmu.2017.01747. eCollection 2017.
66. Kornfeld S F, Biggar K K, Storey K B. Differential expression of mature microRNAs involved in muscle maintenance of hibernating little brown bats, Myotis lucifugus: a model of muscle atrophy resistance. Genomics Proteomics Bioinformatics. 2012 October; 10(5):295-301.
67. Yang T, Chen X L, Huang Z Q, Wen W X, Xu M, Chen D W, Yu B, He J, Luo J Q, Yu J, Mao X B, Zheng P. MicroRNA-27a promotes porcine myoblast proliferation by downregulating myostatin expression. Animal. 2014 November; 8(11):1867-72.
68. Consitt L A, Clark B C The Vicious Cycle of Myostatin Signaling in Sarcopenic Obesity: Myostatin Role in Skeletal Muscle Growth, Insulin Signaling and Implications for Clinical Trials. J Frailty Aging. 2018; 7(1):21-27.
69. Carvalho L P, Basso-Vanelli R P, Di Thommazo-Luporini L, Mendes R G, Oliveira-Junior M C, Vieira R P, Bonjorno-Junior J C, Oliveira C R, Luporini R, Borghi-Silva A. Myostatin and adipokines: The role of the metabolically unhealthy obese phenotype Cytokine in muscle function and aerobic capacity in young adults. 2017 Dec. 12. pii: S1043-4666(17)30379-4. doi:10.1016/j.cyto.2017.12.008.
70. Deng B, Zhang F, Wen J, Ye S, Wang L, Yang Y, Gong P, Jiang S. The function of myostatin in the regulation of fat mass in mammals. Nutr Metab (Lond). 2017 Mar. 21; 14:29. doi: 10.1186/s12986-017-0179-1.
71. Kovanecz I, Gelfand R, Lin G, Sharifzad S, Ohanian A, Ricks R, Lue T, Gonzalez-Cadavid N F. Stem Cells from a Female Rat Model of Type 2 Diabetes/Obesity and Stress Urinary Incontinence Are Damaged by In Vitro Exposure to its Dyslipidemic Serum, Predicting Inadequate Repair Capacity In Vivo. Int. J. Mol. Sci. 2019 Aug. 19; 20 (16):4044.
72. Masouminia M, Gelfand R, Kovanecz I, Vernet D, Tsao J, Salas R, Castro K, Loni L, Rajfer J, Gonzalez-Cadavid N F. Dyslipidemia Is a Major Factor in Stem Cell Damage Induced by Uncontrolled Long-Term Type 2 Diabetes and Obesity in the Rat, as Suggested by the Effects on Stem Cell Culture. J Sex Med. 2018 December; 15(12):1678-1697.

### II. MicroRNAs as Biomarkers of Stem Cell Damage Affecting Autologous Stem Cell Therapy for Erectile Dysfunction and Other Conditions

Introduction. Potential stem cell damage by a disease-induced milieu, and its risks of impaired stem cell therapy, have not virtually been explored so far in either research or therapy. However, it was recently reported that muscle derived stem cells (MDSC) isolated at a late stage diabetes (LD-MDSC) from aged Zucker obese (ZO) rats after their endogenous long-term exposure to untreated type 2 diabetes (T2D) and obesity/dyslipidemia, are severely impaired in their tissue repair ability when implanted into the penile corpora cavernosa of these aged rats, failing to correct the associated penile corporal veno-occlusive dysfunction (CVOD). Moreover, the LD-MDSC were imprinted with a noxious gene global transcriptional phenotype (gene-GTS). This contrasted with the implanted MDSC from young early T2D OZ rats (ED-MDSC). Another study showed that short-term incubation of ED-MDSC with palmitic acid (PA) or cholesterol (CHOL), as representative lipid factors, or with the aged OZ rat dyslipidemic serum, partially replicated the in vivo gene-GTS, caused MDSC fat infiltration, and specifically OZ serum led to overexpression of myostatin, a muscle mass inhibitor/pro-lipofibrotic protein. Objectives. To determine whether these in vivo and in vitro alterations in MDSC are evidenced by changes in the microRNA-GTS (miR-GTS) as a biomarker of stem cell damage, fat infiltration, apoptosis, reduced proliferation, abnormal differentiation, and myostatin upregulation. Methods. ED-MDSC were incubated 4 days in low glucose DMEM/serum, with or without 1-5% aged OZ serum or non-diabetic LZ (lean Zucker) serum, or water soluble 0.5 or 1.0 mM PA, or 50 or 100 mg/dl CHOL, and then subjected to quantitative image analysis (QIA) of fat deposits (Oil red O) and apoptosis (Tunel). RNA was isolated from parallel cultures and from the untreated ED-MDSC and LD-MDSC, and miR-GTS were determined. Results. Fat globules in the in vitro treated ED-MDSC were increased 119.1-, 7.7-, 42.1-, and 60.1-fold, and apoptosis 2.8-, 1.1, 11.3, and 2.8-fold, by OZ serum, LZ serum, PA and cholesterol, respectively. miR-GTS in the in vivo long term exposed LD-MDSC were mostly down-regulated versus the ED-MDSC, specifically by 60-95% in miRs that inhibit myostatin pathways, like miR-27a, miR-199a, and miR-21, as well as in miRs unrelated to myostatin, like miR-99b, miR-100, miR-26a, miR-152, miR-148, let-7f and let-7i. Most of these changes were mimicked in vitro by the ED-MDSC incubation with OZ serum, PA, and CHOL The down-regulated miRs are known to be involved in dyslipidemia, fat infiltration, apoptosis, aberrant stem cell differentiation, inflammation, and senescence. Some miRs are inhibitors of genes reported as over-expressed in vivo in LD-MDSC vs ED-MDSC, such as II-1a, II-6 and II-7 (inflammation), Smad6, Fgf7 and MMPs (fibrosis), and Cxcl1, Cxcl-5, Cxcl16 (inflammatory/fibrotic chemokines). Conclusions. The noxious effects of the T2D/dyslipidemic milieu or factors on MDSC suggest potential risks for autologous stem cell therapy for CVOD in long-term uncontrolled T2D/obese patients. We propose the miR-GTS as a multiple biomarker to identify damaged MDSC, or possibly other stem cells, prior to their therapeutic implantation, or even to detect in the patient blood the presence of endogenous stem cells with poor or abnormal tissue repair capacity.

### EMBODIMENTS

In addition to the various embodiments of the invention described above, embodiments of the invention further include:

1. The diagnosis and follow up of stem cell damage that impairs their capacity for tissue and functional repair, caused by their long-term exposure to the systemic noxious milieu of donor or recipient patients with uncontrolled or poor control type 2 diabetes, metabolic syndrome or obesity, for their application in stem cell therapy.

2. The application of the procedure of Claim 1 for detecting the damaged stem cells, in order to exclude their use as autografts for implanting them back into various organs or tissues of the patient donating their own stem cells for his/her own stem cell therapy, to prevent inadequate efficacy or the differentiation into abnormal differentiated lineages, or any other health complications caused by the stem cell damage.

3. The application of this procedure of Claim 1 for detecting the damaged stem cells, in order to exclude their use for allograft implantation into various organs or tissues of any recipient compatible with the donated stem cells

4. The application of the procedure of Claim 1 for detecting the potential reversal in vitro of the stem cells damage by biological or pharmacological procedures, in order allow for their use for allograft implantation.

5. The application of a procedure that can do the diagnosis and follow up as in Claim 1 that determines the stem cells global transcriptional signatures (miR-GTS) as biomarkers of the stem cell damage, comparing them with the miR-GTS of stem cells isolated from other normal subjects or from cryopreserved specimens isolated when the subject was healthy or free from type 2 diabetes/obesity/dyslipidemia, or from unrelated stem cell banks

6. The same as claim 5, but where only one or several miRs are detected, in either the miR-GTS data, or by procedures to detect specifically individual miRs (miR-ITS) in the stem cells

7. The same as claims 5 and 6 but for foreseeing the damage of the donor stem cells by performing the miR-GTS or miR-ITS determinations in the serum of the donor subjects

8. The same as claim 7 but for foreseeing the damage on any stem cells that would be exposed when implanted to the recipient patient systemic milieu, using the recipient patient's serum

9. The determination of miR-GTS or miR-ITS in the vesicles carrying the miRs (exosomes) isolated from healthy and diabetes/obesity/dyslipidemia damaged stem cells in order to use the healthy donor exosomes therapeutically, by implanting them in vivo in the recipient subject, to prevent endogenous or implanted stem cell damage,

10. The same as Claim 9, but incubating in vitro the healthy subject exosomes with the in vivo damaged stem cells to reverse their damage for their subsequent use for implantation.

11. The application of claims 1 to 10 to diagnostic, follow up, or therapeutic procedures using stem cells, exosomes, or serum, from and in patients having long-term chronic diseases other than uncontrolled diabetes/obesity/dyslipidemia, or physiological noxious conditions as aging, that release cytoquines, chemoquines, reactive oxygen species, likely to alter the miR-GTS and miR-ITS and this be associated with damage to the stem cells and their impairment for stem cell therapy.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of evaluating a stem cell, the method comprising obtaining a microRNA transcriptional phenotype for the stem cell to evaluate the stem cell.

2. The method according to claim 1, wherein the microRNA transcriptional phenotype comprises an expression level for one or more microRNAs.

3. The method according to claim 2, wherein the microRNA transcriptional phonotype comprises an expression level for a plurality of microRNAs.

4. The method according to claim 3, wherein the plurality comprises 5 or more microRNAs.

5. The method according to claim 4, wherein the microRNA transcriptional phenotype comprises a global microRNA transcriptional phenotype.

6. The method according to any of the preceding claims, wherein the evaluating comprises making an assessment of whether the stem cell has been identified and/or damaged.

7. The method according to claim 6, wherein the assessment comprises whether the stem cell has been damaged by the body milieu to which it was exposed prior to isolation and/or afterwards by environmental exposure.

8. The method according to claim 7, wherein the environmental exposure comprises a body source milieu, such as a diabetic and/or obese body source milieu, e.g., type 2 diabetes body source milieu.

9. The method according to claim 7, wherein the environmental exposure comprises a storage condition.

10. The method according to any of the preceding claims, wherein the evaluating comprises making an assessment of the stem cell's therapeutic capacity.

11. A method of evaluating the therapeutic capacity of a stem cell, the method comprising:
obtaining a microRNA transcriptional phenotype for the stem cell; and
using the microRNA transcriptional phenotype to identify its type and assess whether the stem cell is damaged to evaluate the therapeutic capacity of the stem cell.

12. The method according to claim 11, wherein the microRNA transcriptional phenotype comprises an expression level for one or more microRNAs.

13. The method according to claim 12, wherein the microRNA transcriptional phonotype comprises an expression level for a plurality of microRNAs.

14. The method according to claim 13, wherein the plurality comprises 5 or more microRNAs.

15. The method according to claim 14, wherein the microRNA transcriptional phenotype comprises a global microRNA transcriptional phenotype.

16. The method according to any of claims 11 to 15, wherein the method is a method of evaluating the therapeutic capacity of the stem cell for use in the same patient for an autograft procedure.

17. The method according to any of claims 11 to 15, wherein the method is a method of evaluating the therapeutic capacity of the stem cell for use in an allograft procedure.

18. The method according to any of claims 11 to 17, wherein the microRNA transcriptional phenotype is employed to assess whether the stem cell is damaged from its tissue/organ/systemic source milieu.

19. The method according to any of the preceding claims, wherein the stem cell is a mammalian stem cell.

20. The method according to claim 19, wherein the stem cell is a human stem cell.

21. A method of evaluating the therapeutic capacity of a stem cell composition, the method comprising:
obtaining a microRNA transcriptional phenotype for a stem cell of the stem cell composition; and
using the microRNA transcriptional phenotype to assess whether the stem cell is of the stem cell type is claimed and may be damaged, in order to evaluate the therapeutic capacity of the stem cell composition.

22. The method according to claim 21, wherein the microRNA transcriptional phenotype comprises an expression level for one or more microRNAs.

23. The method according to claim 22, wherein the microRNA transcriptional phonotype comprises an expression level for a plurality of microRNAs.

24. The method according to claim 23, wherein the plurality comprises 5 or more microRNAs.

25. The method according to claim 24, wherein the microRNA transcriptional phenotype comprises a global microRNA transcriptional phenotype.

26. The method according to any of claims 21 to 25, wherein the method is a method of evaluating the therapeutic capacity of the stem cell composition for use in an autograft procedure in the same patient.

27. The method according to any of claims 21 to 25, wherein the method is a method of evaluating the therapeutic capacity of the stem cell composition for use in an allograft procedure.

28. The method according to any of claims 21 to 27, wherein the microRNA transcriptional phenotype is employed to assess whether the stem cell composition is damaged from its source milieu.

29. The method according to any of the preceding claims, wherein the stem cell composition is a mammalian stem cell composition.

30. The method according to claim 29, wherein the stem cell composition is a human stem cell composition.

31. The method according to any of claims 21 to 30, wherein the method further comprises administering the stem cell composition to a subject if the stem cell composition is assessed to be undamaged.

32. The method according to any of claims 21 to 30, wherein the method further comprises discarding the stem cell composition if the stem cell composition is assessed to be damaged.

33. The method according to any of claims 21 to 30, wherein the method further comprises contacting the stem cell composition with a restorative agent if the stem cell composition is assessed to be damaged.

34. The method according to claim 33, wherein the restorative agent comprises one or more small molecules.

35. The method according to claim 33, wherein the restorative agent comprises one or more biomolecules.

36. The method according to claim 33, wherein the restorative agent comprises a vesicle.

37. The method according to claim 36, wherein the vesicle comprises an exosome.

38. The method according to claim 37, wherein the method further comprises obtaining the exosome from a healthy donor.

39. The method according to any of claims 33 to 38, wherein the contacting occurs in vitro.

40. The method according to any of claims 33 to 38, wherein the contacting occurs in vivo in the stem cell's receptor patient.

41. A method of evaluating suitability of a subject for a stem cell procedure, the method comprising:
obtaining a microRNA transcriptional phenotype for a sample from the subject; and
comparing the obtained microRNA transcriptional phenotype to a control to evaluate suitability of the subject for the stem cell procedure.

42. The method according to claim 41, wherein the microRNA transcriptional phenotype comprises an expression level for one or more microRNAs.

43. The method according to claim 42, wherein the microRNA transcriptional phonotype comprises an expression level for a plurality of microRNAs.

44. The method according to claim 43, wherein the plurality comprises 5 or more microRNAs.

45. The method according to claim 44, wherein the microRNA transcriptional phenotype comprises a global microRNA transcriptional phenotype.

46. The method according to any of claims 41 to 45, wherein the sample comprises stem cells.

47. The method according to any of claims 41 to 45, wherein the sample comprises exosomes.

48. The method according to any of claims 41 to 45, wherein the sample comprises serum.

49. The method according to any of claims 41 to 48, wherein the subject is a donor in the stem cell procedure.

50. The method according to any of claims 41 to 48, wherein the subject a recipient in the stem cell procedure.

51. A kit for evaluating a stem cell, the kit comprising:
a stem cell microRNA transcriptional phenotype determination component.

52. The kit according to claim 51, wherein the stem cell microRNA transcriptional phenotype comprises an expression level for one or more microRNAs.

53. The kit according to claim 52, wherein the stem cell microRNA transcriptional phonotype comprises an expression level for a plurality of microRNAs.

54. The kit according to claim 53, wherein the plurality comprises 5 or more microRNAs.

55. The kit according to claim 54, wherein the microRNA transcriptional phenotype comprises a global microRNA transcriptional phenotype.

56. The kit according to any of claims 51 to 55, wherein the evaluating comprises making an assessment of whether the stem cell has been damaged.

57. The kit according to claim 56, wherein the assessment comprises whether the stem cell has been damaged by an environmental exposure.

58. The kit according to claim 57, wherein the environmental exposure comprises a source milieu.

59. The kit according to claim 57, wherein the environmental exposure comprises a storage condition.

60. The kit according to any of claims 51 to 59, wherein the evaluating comprises making an assessment of the stem cell's therapeutic capacity.
